(19) United States
(12) Patent Application Publication
(10) Pub. No.: US 2024/0325597 A1
(43) Pub. Date: Oct. 3, 2024

(54) GRAFTED OPHTHALMIC DEVICES CONTAINING DEACTIVATED REGIONS AND PROCESSES FOR THEIR PREPARATION AND USE

(71) Applicant: Johnson & Johnson Vision Care, Inc., Jacksonville, FL (US)

(72) Inventors: Michael F. Widman, Jacksonville, FL (US); Ghulam Maharvi, Jacksonville, FL (US)

(21) Appl. No.: 18/426,780

(22) Filed: Jan. 30, 2024

Related U.S. Application Data
(60) Provisional application No. 63/492,499, filed on Mar. 28, 2023.

(51) Int. Cl. A61L 27/16; A61L 27/52; C08F 2/50; C08F 283/12; C08K 5/5397; G02B 1/04

(52) U.S. Cl. CPC A61L 27/16; A61L 27/52; C08F 2/50; C08F 283/126; C08K 5/5397; G02B 1/043; A61L 2430/16; C08F 2438/00

(57) ABSTRACT

Disclosed are ophthalmic devices formed by a process comprising: (a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker; (b) subjecting the reactive composition to a first activation step such that the reactive composition polymerizes therein to form a crosslinked substrate network containing a covalently bound activatable free radical initiator; (c) deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions; (d) contacting the crosslinked substrate network with a grafting composition containing one or more ethylenically unsaturated compounds, wherein the contacting is conducted under conditions such that the grafting composition penetrates into the crosslinked substrate network; and (e) activating the retained covalently bound activatable free radical initiator such that the grafting composition polymerizes with the crosslinked substrate network, thereby forming grafted polymeric networks, outside of the selective regions and optionally partially within the selective regions.

### RELATED APPLICATIONS

This application claims priority to U.S. Provisional Patent Application Ser. No. 63/492,499, filed Mar. 28, 2023, which is incorporated herein by reference in its entirety.

### FIELD OF THE INVENTION

The invention relates to ophthalmic devices, such as contact lenses, that contain grafted polymeric networks and processes for preparing and using the ophthalmic devices.

### BACKGROUND OF THE INVENTION

The development of polymer materials prepared from individual components that contribute desirable properties is an ongoing goal in many product areas. For instance, polymer materials displaying oxygen permeability and hydrophilicity are desirable for a number of applications within the medical devices field, such as in ophthalmic devices.

A commonly encountered challenge when forming polymeric materials that attempt to combine properties is that in many cases, the individual components from which the final material is made are not readily compatible with each other. For instance, in the contact lens field, silicone hydrogels have been found to provide lenses with significantly increased oxygen permeability and therefore are capable of reducing corneal edema and hyper-vasculature, conditions that may sometimes be associated with conventional hydrogel lenses. Silicone hydrogels have typically been prepared by polymerizing mixtures containing at least one silicone-containing monomer or reactive macromer and at least one hydrophilic monomer. However, silicone hydrogel lenses can be difficult to produce because the silicone components and the hydrophilic components are often incompatible.

In addition to overall compatibility of reactants, it may also be desirable in some applications to form products from different materials, where the materials are localized in certain regions rather than being distributed throughout. For example, in the ophthalmic device field, processes that allow for regional modification of a device (such as a contact lens) may permit manufacturers to create products with customized lens powers or other desirable properties.

### SUMMARY OF THE INVENTION

The invention relates to new polymeric compositions derived from a wide variety of component monomers and polymers, including where such component monomers and polymers are generally incompatible. The invention further relates to processes for selectively modifying the material makeup of a substrate. Such polymeric compositions find use in various applications, for instance, in ophthalmic devices, such as a contact lens, an intraocular lens, a punctal plug, and an ocular insert.

In one aspect, therefore, the invention provides ophthalmic device formed by a process comprising:
(a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
(b) subjecting the reactive composition to a first activation step such that the reactive composition polymerizes therein to form a crosslinked substrate network containing a covalently bound activatable free radical initiator;
(c) deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions;
(d) contacting the crosslinked substrate network with a grafting composition containing one or more ethylenically unsaturated compounds, wherein the contacting is conducted under conditions such that the grafting composition penetrates into the crosslinked substrate network; and
(e) activating the retained covalently bound activatable free radical initiator such that the grafting composition polymerizes with the crosslinked substrate network, thereby forming grafted polymeric networks, outside of the selective regions and optionally partially within the selective regions.

In another aspect, the invention provides an ophthalmic device comprised of a reaction product of a composition comprising:
(a) a crosslinked substrate network, wherein at least a portion of the covalently bound activatable free radical initiators are deactivated in one or more selective regions of the crosslinked substrate network such that the crosslinked substrate network contains retained covalently bound activatable free radical initiators outside of the one or more selective regions and optionally within the one or more selective regions; and
(b) a grafting composition containing one or more ethylenically unsaturated compounds, wherein the grafting composition is localized in the crosslinked substrate network where there are retained covalently bound activatable free radical initiators.

In a further aspect, the invention provides a process for making an ophthalmic device, the process comprising:
(a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
(b) subjecting the reactive composition to a first activation step such that the reactive composition polymerizes therein to form a crosslinked substrate network containing a covalently bound activatable free radical initiator;
(c) deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions;
(d) contacting the crosslinked substrate network with a grafting composition containing one or more ethylenically unsaturated compounds, wherein the contacting is conducted under conditions such that the grafting composition penetrates into the crosslinked substrate network; and
(e) activating the retained covalently bound activatable free radical initiator such that the grafting composition polymerizes with the crosslinked substrate network, thereby forming grafted polymeric networks, outside of the selective regions and optionally partially within the selective regions.

In yet another aspect, the invention provides a process for making an ophthalmic device, the process comprising:
(a) providing a reactive composition containing: (i) a thermal initiator; (ii) one or more ethylenically unsaturated compounds; (iii) a crosslinker; (iv) at least one reactive component having at least one pendant group selected from the group consisting of monoacylphosphine oxide, bisacylphosphine oxide, and combinations thereof; (v) at least one reactive component selected from the group consisting of monoacylphosphine oxide compounds having refractive index or light absorbing moieties, bisacylphosphine oxide compounds having refractive index or light absorbing moieties, and combinations thereof;
(b) subjecting the reactive composition to a thermal polymerization to form a precursor crosslinked substrate network, wherein the monoacylphosphine oxide compounds having refractive index or light absorbing moieties and bisacylphosphine oxide compounds having refractive index or light absorbing moieties are spatially dispersed within the precursor crosslinked substrate network and wherein the reactive composition has not been fully polymerized;
(c) irradiating the precursor crosslinked substrate network in preselected regions, thereby initiating free radical polymerizations from the dispersed monoacylphosphine oxide compounds having refractive index or light absorbing moieties and bisacylphosphine oxide compounds having refractive index or light absorbing moieties, thereby incorporating the refractive index or light absorbing moieties into the precursor crosslinked substrate network in the preselected regions;
(d) subjecting the reactive composition to the thermal polymerization until the reactive composition has been completely polymerized to form a crosslinked substrate network having covalently bound monoacylphosphine oxide or bisacylphosphine oxide groups; and optionally
(e) extracting the unreacted monoacylphosphine oxide compounds having refractive index or light absorbing moieties and bisacylphosphine oxide compounds having refractive index or light absorbing moieties with a solvent;
(f) deactivating at least a portion of the covalently bound monoacylphosphine oxide or bisacylphosphine oxide groups at one or more selective regions of the crosslinked substrate network such that the crosslinked substrate network contains retained covalently bound monoacylphosphine oxide or bisacylphosphine oxide groups outside of the one or more selective regions and optionally within the one or more selective regions;
(g) contacting the crosslinked substrate network with a grafting composition containing one or more ethylenically unsaturated compounds, wherein the contacting is conducted under conditions such that the grafting composition penetrates into the crosslinked substrate network; and
(h) activating the retained covalently bound monoacylphosphine oxide or bisacylphosphine oxide groups such that the grafting composition polymerizes with the crosslinked substrate network, thereby forming grafted polymeric networks, outside of the selective regions and optionally partially within the selective regions; wherein steps (a) through (h) are conducted under conditions that preserve the reactivity of the covalently bound monoacylphosphine oxide or bisacylphosphine oxide groups in the precursor crosslinked substrate network and the crosslinked substrate network until said covalently bound monoacylphosphine oxide or bisacylphosphine oxide groups are intentionally irradiated or activated.

### BRIEF DESCRIPTION OF THE DRAWING

FIG. 1 illustrates a voxel-based lithographic apparatus that may be used to implement some embodiments of the invention.

FIG. 2 depicts an in-mold jig design according to various embodiments of the invention.

FIG. 3 shows the projected images of the deactivation and grafting steps used in Example 1.

FIG. 4 shows micrographs of the deactivated and grafted contact lenses of Example 1.

FIG. 5 depicts one example for carrying a process according to the invention.

### DETAILED DESCRIPTION OF THE INVENTION

Unless defined otherwise, all technical and scientific terms used herein have the same meaning as commonly understood by one of ordinary skill in the art to which the invention belongs. All publications, patent applications, patents, and other references mentioned herein are incorporated by reference.

Unless otherwise indicated, numeric ranges, for instance as in “from 2 to 10” or as in “between 2 and 10” are inclusive of the numbers defining the range (e.g., 2 and 10).

Unless otherwise indicated, ratios, percentages, parts, and the like are by weight.

The phrase “number average molecular weight” refers to the number average molecular weight (Mₙ) of a sample; the phrase “weight average molecular weight” refers to the weight average molecular weight (M_{w}) of a sample; the phrase “polydispersity index” (PDI) refers to the ratio of M_{w} divided by Mₙ and describes the molecular weight distribution of a sample. If the type of “molecular weight” is not indicated or is not apparent from the context, then it is intended to refer to number average molecular weight.

As used herein, the term “about” refers to a range of +/−10 percent of the number that is being modified. For example, the phrase “about 10” would include both 9 and 11.

As used herein, the term “(meth)” designates optional methyl substitution. Thus, a term such as “(meth)acrylate” denotes both methacrylate and acrylate.

Wherever chemical structures are given, it should be appreciated that alternatives disclosed for the substituents on the structure may be combined in any combination. Thus, if a structure contained substituents R* and R**, each of which contained three lists of potential groups, 9 combinations are disclosed. The same applies for combinations of properties.

The average number of repeating units in a polymer sample is known as its “degree of polymerization.” When a generic chemical formula of a polymer sample, such as [***]ₙ is used, “n” refers to its degree of polymerization, and the formula shall be interpreted to represent the number average molecular weight of the polymer sample.

As used herein, the term “individual” includes humans and vertebrates.

As used herein, the term “ophthalmic device” refers to any device which resides in or on the eye or any part of the eye, including the ocular surface. These devices can provide optical correction, cosmetic enhancement, vision enhancement, therapeutic benefit (for example as bandages) or delivery of active components such as pharmaceutical and nutraceutical components, or a combination of any of the foregoing. Examples of ophthalmic devices include but are not limited to lenses, optical and ocular inserts, including but not limited to punctal plugs, and the like. “Lenses” include soft contact lenses, hard contact lenses, hybrid contact lenses, intraocular lenses, and inlay and overlay lenses. The ophthalmic device preferably may comprise a contact lens.

As used herein, the term “contact lens” refers to an ophthalmic device that can be placed on the cornea of an individual's eye. The contact lens may provide corrective, cosmetic, or therapeutic benefit, including wound healing, the delivery of drugs or nutraceuticals, diagnostic evaluation or monitoring, ultraviolet light blocking, visible light or glare reduction, or any combination thereof. A contact lens can be of any appropriate material known in the art and can be a soft lens, a hard lens, or a hybrid lens containing at least two distinct portions with different physical, mechanical, or optical properties, such as modulus, water content, light transmission, or combinations thereof.

The ophthalmic devices and contact lenses of the invention may be comprised of silicone hydrogels. These silicone hydrogels typically contain at least one hydrophilic monomer and at least one silicone-containing component that are covalently bound to one another in the cured device. The ophthalmic devices and contact lenses of the invention may also be comprised of conventional hydrogels, or combination of conventional and silicone hydrogels.

A “macromolecule” is an organic compound having a number average molecular weight of greater than 1500 grams/mole and may be reactive or non-reactive.

As used herein, the “target macromolecule” is the intended macromolecule being synthesized from the reactive composition comprising monomers, macromers, prepolymers, cross-linkers, initiators, additives, diluents, and the like.

As used herein, a “monomer” is a mono-functional molecule which can undergo chain growth polymerization, and in particular, free radical polymerization, thereby creating a repeating unit in the chemical structure of the target macromolecule. Some monomers have di-functional impurities that can act as cross-linking agents. A “hydrophilic monomer” is also a monomer which yields a clear single-phase solution when mixed with deionized water at 25° C. at a concentration of 5 weight percent. A “hydrophilic component” is a monomer, macromer, prepolymer, initiator, cross-linker, additive, or polymer which yields a clear single-phase solution when mixed with deionized water at 25° C. at a concentration of 5 weight percent.

As used herein, a “macromonomer” or “macromer” is a linear or branched macromolecule having at least one polymerizable group that can undergo chain growth polymerization, and in particular, free radical polymerization.

As used herein, the term “polymerizable” means that the compound comprises at least one polymerizable group. “Polymerizable groups” are groups that can undergo chain growth polymerization, such as free radical and/or cationic polymerization, for example a carbon-carbon double bond group which can polymerize when subjected to radical polymerization initiation conditions. Non-limiting examples of polymerizable groups (Rg) include (meth)acrylates, styrenes, vinyl ethers, (meth)acrylamides, N-vinyllactams, N-vinylamides, O-vinylcarbamates, O-vinylcarbonates, and other vinyl groups. Preferably, the polymerizable groups comprise (meth)acrylates, (meth)acrylamides, and mixtures thereof. Preferably, the polymerizable groups comprise (meth)acrylate, (meth)acrylamide, N-vinyl lactam, N-vinylamide, styryl functional groups, or mixtures of any of the foregoing. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted). In contrast to “polymerizable,” the term “non-polymerizable” means that the compound does not comprise such a free radical polymerizable group.

Examples of the foregoing include substituted or unsubstituted C₁₋₆alkyl(meth)acrylates, C₁₋₆alkyl(meth)acrylamides, C₂₋₁₂alkenyls, C₂₋₁₂alkenylphenyls, C₂₋₁₂alkenylnaphthyls, C₂₋₆alkenylphenylC₁₋₆alkyls, where suitable substituents on said C₁₋₆ alkyls include ethers, hydroxyls, carboxyls, halogens and combinations thereof.

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

An “ethylenically unsaturated compound” is a monomer, macromer, or prepolymer that contains at least one polymerizable group. An ethylenically unsaturated compound may preferably consist of one polymerizable group.

As used herein, a “silicone-containing component” or “silicone component” is a monomer, macromer, prepolymer, cross-linker, initiator, additive, or polymer in the reactive composition with at least one silicon-oxygen bond, typically in the form of siloxy groups, siloxane groups, carbosiloxane groups, and mixtures thereof. Examples of silicone-containing components which are useful in this invention may be found in U.S. Pat. Nos. 3,808,178, 4,120,570, 4,136,250, 4,153,641, 4,740,533, 5,034,461, 5,070,215, 5,244,981, 5,314,960, 5,331,067, 5,371,147, 5,760,100, 5,849,811, 5,962,548, 5,965,631, 5,998,498, 6,367,929, 6,822,016, 6,943,203, 6,951,894, 7,052,131, 7,247,692, 7,396,890, 7,461,937, 7,468,398, 7,538,146, 7,553,880, 7,572,841, 7,666,921, 7,691,916, 7,786,185, 7,825,170, 7,915,323, 7,994,356, 8,022,158, 8,163,206, 8,273,802, 8,399,538, 8,415,404, 8,420,711, 8,450,387, 8,487,058, 8,568,626, 8,937,110, 8,937,111, 8,940,812, 8,980,972, 9,056,878, 9,125,808, 9,140,825, 9,156,934, 9,170,349, 9,217,813, 9,244,196, 9,244,197, 9,260,544, 9,297,928, 9,297,929, and European Patent No. 080539. These patents are hereby incorporated by reference in their entireties.

A “polymer” is a target macromolecule composed of the repeating units of the monomers and macromers used during polymerization.

A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

A “repeating unit” is the smallest group of atoms in a polymer that corresponds to the polymerization of a specific monomer or macromer.

An “initiator” is a molecule that can decompose into free radical groups which can react with a monomer to initiate a free radical polymerization reaction. A thermal initiator decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A photo-initiator decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “free radical group” is a molecule that has an unpaired valence electron which can react with a polymerizable group to initiate a free radical polymerization reaction.

A “cross-linking agent” or “crosslinker” is a di-functional or multi-functional monomer which can undergo free radical polymerization at two or more locations on the molecule, thereby creating branch points and a polymeric network. The two or more polymerizable functionalities on the crosslinker may be the same or different and may, for instance, be independently selected from vinyl groups (including allyl), (meth)acrylate groups, and (meth)acrylamide groups. Common examples are ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers (or macromers) which contains remaining polymerizable groups capable of undergoing further reaction to form a polymer.

A “polymeric network” is a type of polymer that is in the form of a cross-linked macromolecule. Generally, a polymeric network may swell but cannot dissolve in solvents. For instance, the crosslinked substrate network of the invention is a material that is swellable, without dissolving.

“Hydrogels” are polymeric networks that swell in water or aqueous solutions, typically absorbing at least 10 weight percent water (at 25° C.). “Silicone hydrogels” are hydrogels that are made from at least one silicone-containing component with at least one hydrophilic component. Hydrophilic components may also include non-reactive polymers.

“Conventional hydrogels” refer to polymeric networks made from monomers without any siloxy, siloxane or carbosiloxane groups. Conventional hydrogels are prepared from reactive compositions predominantly containing hydrophilic monomers, such as 2-hydroxyethyl methacrylate (“HEMA”), N-vinyl pyrrolidone (“NVP”), N, N-dimethylacrylamide (“DMA”) or vinyl acetate.

As used herein, the term “reactive composition” refers to a composition containing one or more reactive components (and optionally non-reactive components) which are mixed (when more than one is present) together and, when subjected to polymerization conditions, form polymer compositions. If more than one component is present, the reactive composition may also be referred to herein as a “reactive mixture” or a “reactive monomer mixture” (or RMM).

The reactive composition comprises reactive components such as the monomers, macromers, prepolymers, cross-linkers, and initiators, and optional additives such as wetting agents, release agents, dyes, light absorbing compounds such as UV-VIS absorbers, pigments, dyes and photochromic compounds, any of which may be reactive or non-reactive but are preferably capable of being retained within the resulting polymer composition, as well as pharmaceutical and nutraceutical compounds, and any diluents. It will be appreciated that a wide range of additives may be added based upon the final product which is made and its intended use. Concentrations of components of the reactive composition are expressed as weight percentages of all components in the reaction composition, excluding diluent. When diluents are used, their concentrations are expressed as weight percentages based upon the amount of all components in the reaction composition and the diluent.

“Reactive components” are the components in the reactive composition which become part of the chemical structure of the resulting material by covalent bonding, hydrogen bonding, electrostatic interactions, the formation of interpenetrating polymeric networks, or any other means. Examples include, but are not limited, to silicone reactive components (e.g., the silicone-containing components described below) and hydrophilic reactive components (e.g., the hydrophilic monomers described below).

As used herein, the term “silicone hydrogel contact lens” refers to a contact lens comprising at least one silicone hydrogel. Silicone hydrogel contact lenses generally have increased oxygen permeability compared to conventional hydrogels. Silicone hydrogel contact lenses use both their water and polymer content to transmit oxygen to the eye.

“DMD” refers to a digital micromirror device that may be a bistable spatial light modulator consisting of an array of movable micromirrors functionally mounted over a CMOS SRAM. Each mirror may be independently controlled by loading data into the memory cell below the mirror to steer reflected light, spatially mapping a pixel of video data to a pixel on a display. The data electrostatically controls the mirror's tilt angle in a binary fashion, where the mirror states are either +X degrees (on) or −X degrees (off). Light reflected by the on mirrors then may be passed through a projection lens and onto a screen. Light may be reflected off to create a dark field, and defines the black-level floor for the image. Images may be created by gray-scale modulation between on and off levels at a rate fast enough to be integrated by the observer. The DMD (digital micromirror device) may comprise DLP projection systems.

“DMD Script” refers to a control protocol for a spatial light modulator and also to the control signals of any system component, for example, a light source or filter wheel, either of which may include a series of command sequences in time.

The term “multi-functional” refers to a component having two or more polymerizable groups. The term “mono-functional” refers to a component having one polymerizable group.

The terms “halogen” or “halo” indicate fluorine, chlorine, bromine, and iodine.

As used herein, the term “alkyl” refers to an unsubstituted or substituted linear or branched alkyl group containing the indicated number of carbon atoms. If no number is indicated, then alkyl (optionally including any substituents on alkyl) may contain 1 to 16 carbon atoms. Preferably, the alkyl group contains 1 to 10 carbon atoms, alternatively 1 to 7 carbon atoms, or alternatively 1 to 4 carbon atoms. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, amido, carbamate, carbonate, halogen, phenyl, benzyl, and combinations thereof. “Alkylene” means a divalent alkyl group, such as —CH₂—, —CH₂CH₂—, —CH₂CH₂CH₂—, —CH₂CH(CH₃)CH₂—, and —CH₂CH₂CH₂CH₂—.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, more preferably 1-4 carbons, and still more preferably 1-2 carbons. “Haloalkyl” includes perhaloalkyl groups, such as —CF₃— or —CF₂CF₃—. “Haloalkylene” means a divalent haloalkyl group, such as —CH₂CF₂—.

“Cycloalkyl” refers to an unsubstituted or substituted cyclic hydrocarbon containing the indicated number of ring carbon atoms. If no number is indicated, then cycloalkyl may contain 3 to 12 ring carbon atoms. Preferred are C₃-C₈ cycloalkyl groups, more preferably C₄-C₇ cycloalkyl, and still more preferably C₅-C₆ cycloalkyl. Examples of cycloalkyl include cyclopropyl, cyclobutyl, cyclopentyl, cyclohexyl, cycloheptyl and cyclooctyl. Examples of substituents on cycloalkyl include 1, 2, or 3 groups independently selected from alkyl, hydroxy, amino, amido, oxa, carbonyl, alkoxy, amido, carbamate, carbonate, halo, phenyl, benzyl, and combinations thereof. “Cycloalkylene” means a divalent cycloalkyl group, such as 1,2-cyclohexylene, 1,3-cyclohexylene, or 1,4-cyclohexylene.

“Heterocycloalkyl” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. Heterocycloalkylene means a divalent heterocycloalkyl group.

“Aryl” refers to an unsubstituted or substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, amido, carbamate, carbonate, halo, phenyl, benzyl, and combinations thereof. “Arylene” means a divalent aryl group, for example 1,2-phenylene, 1,3-phenylene, or 1,4-phenylene.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Alkoxy” refers to an alkyl group attached to the parent molecular moiety through an oxygen bridge. Examples of alkoxy groups include, for instance, methoxy, ethoxy, propoxy and isopropoxy. “Aryloxy” refers to an aryl group attached to a parent molecular moiety through an oxygen bridge. Examples include phenoxy. “Cyclic alkoxy” means a cycloalkyl group attached to the parent moiety through an oxygen bridge.

“Alkylamine” refers to an alkyl group attached to the parent molecular moiety through an —NH bridge. Alkyleneamine means a divalent alkylamine group, such as —CH₂CH₂NH—.

“Siloxanyl” refers to a structure having at least one Si—O—Si bond. Thus, for example, siloxanyl group means a group having at least one Si—O—Si group (i.e., a siloxane group), and siloxanyl compound means a compound having at least one Si—O—Si group. “Siloxanyl” encompasses monomeric (e.g., Si—O—Si) as well as oligomeric/polymeric structures (e.g., —[Si—O]ₙ—, where n is 2 or more). Each silicon atom in the siloxanyl group is substituted with independently selected R^{A} groups (where R^{A} is as defined in formula A options (b)-(i)) to complete their valence.

“Silyl” refers to a structure of formula R₃Si— and “siloxy” refers to a structure of formula R₃Si—O—, where each R in silyl or siloxy is independently selected from trimethylsiloxy, C₁-C₈ alkyl (preferably C₁-C₃ alkyl, more preferably ethyl or methyl), and C₃-C₈ cycloalkyl.

“Alkyleneoxy” refers to groups of the general formula -(alkylene-O)ₚ— or —(O-alkylene)ₚ-, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH₂CH₂O]ₚ— or CH₃O—[CH₂CH₂O]ₚ—). Examples of alkyleneoxy include polymethyleneoxy, polyethyleneoxy, polypropyleneoxy, polybutyleneoxy, and poly(ethyleneoxy-co-propyleneoxy).

“Oxaalkylene” refers to an alkylene group as defined above where one or more non-adjacent CH₂ groups have been substituted with an oxygen atom, such as —CH₂CH₂OCH(CH₃)CH₂—. “Thiaalkylene” refers to an alkylene group as defined above where one or more non-adjacent CH₂ groups have been substituted with a sulfur atom, such as —CH₂CH₂SCH(CH₃)CH₂—.

The term “linking group” refers to a moiety that links the polymerizable group to the parent molecule. The linking group may be any moiety that does not undesirably interfere with the polymerization of the compound of which it is a part. For instance, the linking group may be a bond, or it may comprise one or more alkylene, haloalkylene, amide, amine, alkyleneamine, carbamate, carboxylate (—CO₂—), arylene, heteroarylene, cycloalkylene, heterocycloalkylene, alkyleneoxy, oxaalkylene, thiaalkylene, haloalkyleneoxy (alkyleneoxy substituted with one or more halo groups, e.g., —OCF₂—, —OCF₂CF₂—, —OCF₂CH₂—), siloxanyl, alkylenesiloxanyl, or combinations thereof. The linking group may optionally be substituted with 1 or more substituent groups. Suitable substituent groups may include those independently selected from alkyl, halo (e.g., fluoro), hydroxyl, HO-alkyleneoxy, MeO-alkyleneoxy, siloxanyl, siloxy, siloxy-alkyleneoxy-, siloxy-alkylene-alkyleneoxy- (where more than one alkyleneoxy groups may be present and wherein each methylene in alkylene and alkyleneoxy is independently optionally substituted with hydroxyl), ether, amine, carbonyl, carbamate, and combinations thereof. The linking group may also be substituted with a polymerizable group, such as (meth)acrylate.

Preferred linking groups include C₁-C₈ alkylene (preferably C₂-C₆ alkylene) and C₁-C₈ oxaalkylene (preferably C₂-C₆ oxaalkylene), each of which is optionally substituted with 1 or 2 groups independently selected from hydroxyl and siloxy. Preferred linking groups also include carboxylate, amide, C₁-C₈ alkylene-carboxylate-C₁-C₈ alkylene, or C₁-C₈ alkylene-amide-C₁-C₈ alkylene.

When the linking group is comprised of combinations of moieties as described above (e.g., alkylene and cycloalkylene), the moieties may be present in any order. For instance, if in Formula A below, L is indicated as being -alkylene-cycloalkylene-, then Rg-L may be either Rg-alkylene-cycloalkylene-, or Rg-cycloalkylene-alkylene-. Notwithstanding this, the listing order represents the preferred order in which the moieties appear in the compound starting from the terminal polymerizable group (Rg or Pg) to which the linking group is attached. For example, if in Formula A, L is indicated as being alkylene-cycloalkylene, then Pg-L is preferably Pg-alkylene-cycloalkylene-.

As noted above, in one aspect, the invention provides an ophthalmic device formed by a process comprising: (a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker; (b) subjecting the reactive composition to a first activation step such that the reactive composition polymerizes therein to form a crosslinked substrate network containing a covalently bound activatable free radical initiator; (c) deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions; (d) contacting the crosslinked substrate network with a grafting composition containing one or more ethylenically unsaturated compounds, wherein the contacting is conducted under conditions such that the grafting composition penetrates into the crosslinked substrate network; and (e) activating the retained covalently bound activatable free radical initiator such that the grafting composition polymerizes with the crosslinked substrate network, thereby forming grafted polymeric networks, outside of the selective regions and optionally partially within the selective regions.

In another aspect, the invention provides an ophthalmic device comprised of a reaction product of a composition comprising: (a) a crosslinked substrate network, wherein at least a portion of the covalently bound activatable free radical initiators are deactivated in one or more selective regions of the crosslinked substrate network such that the crosslinked substrate network contains retained covalently bound activatable free radical initiators outside of the one or more selective regions and optionally within the one or more selective regions; and (b) a grafting composition containing one or more ethylenically unsaturated compounds, wherein the grafting composition is localized in the crosslinked substrate network where there are retained covalently bound activatable free radical initiators.

The polymerization initiator may be any composition with the ability to generate free radical groups in two or more separate activation steps. There is no particular requirement in the invention with respect to what type of polymerization initiator is used or the mechanism of activation, as long as the first activation and the second activation can be conducted sequentially. Thus, suitable polymerization initiators may, for example, be activated thermally, by visible light, by ultraviolet light, via electron beam irradiation, by gamma ray irradiation, or combinations thereof. Examples of polymerization initiators for use in the invention include, without limitation, bisacylphosphine oxides (“BAPO”), bis(acyl)phosphane oxides (e.g., bis(mesitoyl)phosphinic acid), azo compounds, peroxides, alpha-hydroxy ketones, alpha-alkoxy ketones, 1, 2-diketones, germanium based compounds (such as bis(4-methoxybenzoyl)diethylgermanium), or combinations thereof. More specifically, the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

BAPO initiators are preferred. Examples of suitable BAPO initiators include, without limitation, compounds having the chemical structure of formula I:

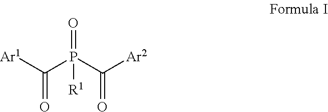

wherein Ar¹ and Ar² are independently substituted or unsubstituted aryl groups, typically substituted phenyl groups, wherein the substituents are linear, branched, or cyclic alkyl groups, such as methyl groups, linear, branched, or cyclic alkoxy groups, such as methoxy groups, and halogen atoms; preferably Ar¹ and Ar² have identical chemical structures; and wherein R¹ is a linear, branched, or cyclic alky group having from 1 to 10 carbon atoms, or R¹ is a phenyl group, a hydroxyl group, or an alkoxy group having from 1 to 10 carbon atoms.

It should be noted that polymerization initiators that are activatable by different types of energy for the initial and subsequent activations may be used. For instance, materials that undergo a first thermal activation and a second activation via irradiation are within the scope of the invention. Examples of such mixed activation materials include compounds of formulae II, III, IV, and V:

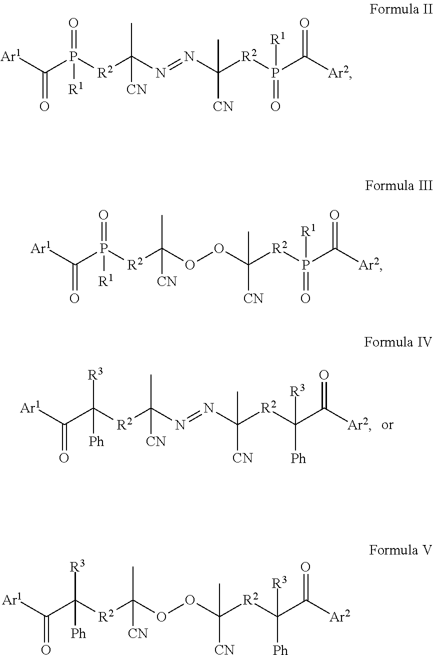

wherein Ar¹ and Ar² are independently substituted or unsubstituted aryl groups, typically substituted phenyl groups, wherein the substituents are linear, branched, or cyclic alkyl groups, such as methyl groups, linear, branched, or cyclic alkoxy groups, such as methoxy groups, and halogen atoms; preferably Ar¹ and Ar² have identical chemical structures; and wherein R¹ is a linear, branched, or cyclic alkyl group having from 1 to 10 carbon atoms; wherein R² is difunctional methylene linking group that may further comprise ether, ketone, or ester groups along the methylene chain having from 1 to 10 carbon atoms; and R³ is a hydrogen atom, a hydroxyl group, or a linear, branched, or cyclic alkoxy group having from 1 to 10 carbon atoms. A further example is tert-butyl 7-methyl-7-(tert-butylazo)peroxyoctanoate.

Furthermore, diazo compounds, diperoxy compounds, or azo-peroxy compounds that exhibit two distinct decomposition temperatures may be used in the prevent invention.

Preferably, the polymerization initiator is a photopolymerization initiator, preferably a bisacylphosphine oxide. Bisacylphosphine oxides are desirable because they can undergo sequential activations steps at different wavelengths and are therefore simple to use. At the longer wavelength, bisacylphosphine oxides can form two free radical groups, one of which is a monoacylphosphine oxide. The monacylphosphine oxide (MAPO) can then undergo a second activation, typically at a shorter wavelength. A particularly preferred bisacylphosphine oxide is bis(2,4,6-trimethylbenzoyl) phenylphosphine oxide, for which the longer wavelength is typically above 420 nm (e.g., 435 nm and above) and the shorter wavelength is typically 420 nm and below. It may be preferable to use an LED or equivalent light in which the bandwidths are relatively narrow as the radiation source, thereby allowing initial irradiation while preserving some or most of the MAPO groups in the crosslinked substrate network.

Other exemplary bisacylphosphine oxide compounds that may be used include bis-(2,6-dimethoxybenzoyl)-2,4,4-trimethylpenthylphosphine oxide, bis(2,4,6-trimethylbenzoyl)-2,4,4-trimethylpenthylphosphine oxide, or bis(2,4,6-trimethylbenzoyl)phosphinic acid or salt thereof.

In the invention, the reactive composition, which contains the polymerization initiator, one or more ethylenically unsaturated compounds, and a crosslinker, is subjected to a first activation step under conditions that cause the polymerization initiator to undergo its initial activation. For example, if the polymerization initiator is a BAPO, the reactive composition may be irradiated at 435 nm or above using an appropriate light source. The reactive composition consequently polymerizes to form a crosslinked substrate network. The crosslinked substrate network contains the residue of the polymerization initiator as a covalently bound activatable free radical initiator.

The activation and polymerization of the reactive composition may be carried out using techniques known to those skilled in the art. For example, the reactive components of the reactive composition may be mixed in a vessel. A diluent may optionally be used to facilitate the mixing. The mixture may be filtered, degassed, and heated to a desired temperature and then irradiated under conditions to cause a first activation of the polymerization initiator and consequent formation of the crosslinked substrate network. The vessel for the polymerization may be a mold, for instance where it is desired for the product to have a specific shape. For example, the reactive composition may be dosed and polymerized within the cavity of a mold pair (e.g., front and back molds). Preferably, the crosslinked substrate network for use in ophthalmic devices of the invention is a conventional or a silicone hydrogel. More preferably, it is a silicone hydrogel.

Alternatively, the crosslinked substrate network is formed by a thermal polymerization of a reactive composition comprising at least one ethylenically unsaturated compound; at least one reactive component selected from the group consisting of a monoacylphosphine oxide compound, a bisoacylphosphine oxide compound, and combinations thereof; and a thermal crosslinker. Examples of a thermal initiator include, without limitation, azo compounds such as azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A preferred thermal initiator is azobisisobutyronitrile. Thermal polymerization reactions are typically carried out at temperatures between 50° C. and 150° C., preferably between 50° C. and 125° C., and most preferably between 60° C. and 100° C.

One type of monoacylphosphine oxide compound is a monoacylphosphine oxide monomer (MAPO-M) containing a polymerizable group and a monoacylphosphosine oxide group. MAPO-M may have many different chemical structures. Examples include Formula MAPO-M1: Rg-L-PO(R)(COAr¹) and Formula MAPO-M2: Rg-L-CO—POAr1Ar2, wherein Rg is a polymerizable group, L is a linking group (including a direct bond), and Ar¹ and Ar² are independently aryl groups which may have substituents. Forming a crosslinked substrate network by a thermal polymerization of a reactive composition comprising MAPO-M components produces a crosslinked substrate network with monoacylphosphine oxide groups as the covalently bound activatable free radical initiators. As disclosed in Biomacromolecules 2001, 2, 1271-1278, one example of such a monoacylphosphine oxide monomer is (diphenylphophosyl)-(4-vinylphenyl)-methanone (DPPM) having the chemical structure shown below:

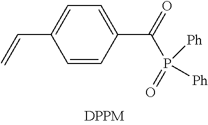 [SMILES: C=Cc1ccc(C(=O)P(=O)(c2ccccc2)c2ccccc2)cc1]

One type of bisacylphosphine oxide compound is a bisacylphosphine oxide monomer (BAPO-M) containing a polymerizable group and a bisacylphosphosine oxide group. As disclosed in U.S. Pat. No. 8,883,872, which is incorporated herein by reference in its entirety, an exemplary BAPO-M may be depicted in the Formula BAPO-M1: Rg-L-PO(COAr¹)(COAr²), wherein Rg is a polymerizable group, L is a linking group (including a direct bond), and Ar¹ and Ar² are independently aryl groups which may have substituents. Forming a crosslinked substrate network by a thermal polymerization of a reactive composition comprising BAPO-M components produces a crosslinked substrate network with bisacylphosphine oxide groups as the covalently bound activatable free radical initiators. One example of a BAPO-M is ethyl 2-(bis[2,4,6-trimethylbenzoyl]-phosphoryl)methacrylate. One advantage of using MAPO-M in the reactive composition to form the covalently bound activatable free radical initiator in the crosslinked substrate network is control over the concentration of monoacylphosphine oxide groups for subsequent deactivation and grafting. The inclusion of BAPO-M in the reactive composition enables even more possible combinations of grafting and deactivation. As an illustration only, but not be construed as limiting the full scope of options in any way, an ophthalmic device may be fabricated using the following process: (1) grafting a first grafting composition into first selected regions of a BAPO-M crosslinked substrate network using a wavelength of light that does not activate the MAPO groups formed thereby, for instance by irradiating with 435 nanometer light; (2) deactivating the resulting MAPO groups and any residual BAPO groups by irradiating with 405 nanometer light in second selected regions; and then (3) grafting a second grafting composition in third selected regions of a MAPO crosslinked substrate network, which may or may not overlap with the first selected regions or second selected regions, depending on the levels of deactivation and the locations of the selected regions used in each step.

As another illustration, but not to be construed as limiting the full scope of options in any way, an ophthalmic device may be fabricated using the following process: (1) convertingting BAPO groups into MAPO groups in first selected regions of a thermally cured BAPO-M crosslinked substrate network using a wavelength of light that does not activate the MAPO groups formed thereby, for instance, by irradiating with 435 nanometer light; (2) deactivating both MAPO and BAPO groups in second selected regions, for instance, by irradiating with 405 nanometer light in second selected regions, said second selected regions may optionally overlap with the first selected regions; (3) grafting a first grafting composition in un-deactivated (BAPO containing) regions, for instance, by irradiating with 435 nanometer light; and (4) grafting a second grafting composition in the first selected (MAPO containing) regions, for instance, by irradiating at 405 nanometers. No grafting occurs in the second selected regions. If the first grafting composition contains a reactive dye (D1) and the second grafting composition contains another reactive dye (D2), then by varying the irradiation intensity profiles in any or all of the aforementioned steps (1) through (4) as well as the relative amounts of D1 and D2 in the grafting compositions, a wide range of apodization patterns can be fabricated into the ophthalmic device. Said apodization patterns can be formed in the optical zone to augment the optics of the ophthalmic lens or in the periphery to modify the appearance of the iris when the ophthalmic device is a contact lens. Before or in-between the above grafting steps (3) and (4), the crosslinked substrate network can be extracted and/or hydrated.

Another type of monoacylphosphine oxide compound is a monoacylphosphine oxide compound having refractive index (MAPO-RI) or light absorbing (MAPO-LA) moieties as disclosed in U.S. patent application Ser. No. 17/821,311 which is hereby incorporated by reference in its entirety. Similarly, another type of bisacylphosphine oxide compound is a bisacylphosphine oxide compound having refractive index (BAPO-RI) or light absorbing (BAPO-LA) moieties as disclosed in U.S. patent application Ser. No. 17/821,311 which is hereby incorporated by reference in its entirety. These MAPO-RI, MAPO-LA, BAPO-RI, and BAPO-LA compounds may be incorporated into the crosslinked substrate network by a combination of thermal polymerization and photochemical having the following steps: (a) thermally polymerizing the reactive composition, comprising at least one ethylenically unsaturated compound, at least one reactive component selected from the group consisting of a monoacylphosphine oxide monomer (MAPO-M), a bisacylphosphine oxide monomer (MAPO-M), or combinations thereof; and a thermal crosslinker, to form a precursor crosslinked substrate network, wherein the monoacylphosphine oxide compounds having refractive index or light absorbing moieties and bisacylphosphine oxide compounds having refractive index or light absorbing moieties are spatially dispersed within a precursor crosslinked substrate network and wherein the reactive composition has not been fully polymerized; (b) irradiating the precursor crosslinked substrate network in preselected regions, thereby initiating the free radical polymerization from the dispersed monoacylphosphine oxide compounds having refractive index or light absorbing moieties and bisacylphosphine oxide compounds having refractive index or light absorbing moieties, thereby incorporating the refractive index or light absorbing moieties into the precursor crosslinked substrate network; (c) thermally polymerizing until the reactive composition has been completely polymerized; and optionally (d) extracting the unreacted monoacylphosphine oxide compounds having refractive index or light absorbing moieties and bisacylphosphine oxide compounds having refractive index or light absorbing moieties with a solvent; and wherein steps (a) through (d) are conducted under conditions that preserve the reactivity of the repeating units derived from the reactive components having at least one pendant group selected from the group consisting of monoacylphosphine oxide, bisacylphosphine oxide, and combinations thereof in the precursor crosslinked substrate network and the crosslinked substrate network.

Incorporating MAPO-RI, MAPO-LA, BAPO-RI, and BAPO-LA compounds into the precursor crosslinked substrate network after partial thermal cure and subsequently into the crosslinked substrate network upon full thermal cure may be designed to affect an overall change in the optical path length with or without a concomitant change in the absorption spectrum in the preselected regions of the crosslinked substrate network. The MAPO-RI, MAPO-LA, BAPO-RI, and BAPO-LA compounds are spatially dispersed in the precursor crosslinked substrate network after partial thermal cure in terms of their concentration based on solubility and cure kinetics including gelation and phase morphology. Upon irradiation, the MAPO-RI, MAPO-LA, BAPO-RI, and BAPO-LA compounds copolymerize with reactive composition components in the preselected regains. In this way, the overall optics of the lens can be modified in a deterministic manner. The optical path length (OPL) is calculated by the formula: OPL=∫ₐ^{b}n(s)ds where n(s) is refractive index as a function of distance travelled through a medium (s) where the light path travels between points a and b (see Field Guide to Geometrical Optics, John E. Greivenkamp, Editor, University of Arizona, SPIE Field Guides, Volume FG01, SPIE Press, Bellingham WA, USA, 2004). For homogeneous media, OPL is simply the refractive index times the distance travelled (n times s). In theory, the OPL can be modified by the incorporation of a functional moiety by either changing the refractive index of the region, by changing the distance travelled by light rays through the region, for instance, by changing the swelling properties of the region based on compositional and/or crosslink density modifications which in turn affect the thickness profile of the optical zone of the lens, or any combination of factors that change the OPL in the region. In this application, the term “refractive index moiety” is defined as a functional moiety that changes the OPL in part by changing the refractive index of the preselected regions. The composition of the reactive monomer mixture will determine the impact of swelling and/or crosslink density on the OPL of the preselected regions. In this application, the term “light absorbing moiety” is defined as a functional moiety that changes the light absorbing properties or spectra of the preselected regions.

The functional moiety may, for instance, be a refractive index moiety, a light absorbing moiety, or a combination thereof. Light absorbing moieties may be used to provide an ophthalmic lens with a variety of functions including, for instance, cosmetic features, or the absorption of specific wavelengths of light (e.g., absorption of high energy visible (HEV) light and/or other wavelengths). The functional moiety may provide more than one function. For instance, a light absorbing moiety may also modify the refractive index of the lens in a desirable manner. Thus, incorporated moieties may achieve multiple effects.

Light absorbing moieties may be used to provide general or custom apodization features to the lens in order to improve a lens user's vision. Examples of light absorbing moieties include, for instance, static dyes, photochromic dyes, thermochromic dyes, leuco dyes, and combinations thereof. More specific examples include, without limitation: azo-based dyes; anthraquinone-based dyes; nitro-based dyes; phthalocyanine-based dyes; quinoneimine-based dyes; quinoline-based dyes; carbonyl-based dyes; triarylmethane-based dyes; methine-based dyes; naphthopyrans; spiro (indoline) quinopyrans and spiro (indoline) pyrans; oxazines, such as spiro (indoline) naphthoxazines, spiro (indoline) pyridobenzoxazines, spiro (benzindoline) pyridobenzoxazines, spiro (benzindoline) naphthoxazines and spiro (indoline) benzoxazines; mercury dithizonates; fulgides; fulgimides, acridine dyes, arylmethane dyes, indamin, xanthene, acridone, or combinations thereof.

The functional moiety may be a refractive index moiety. A refractive index moiety alters the OPL of the lens, in the areas where it is incorporated, relative to the bulk lens. Variations in refractive properties across a lens can be used to impart images or other visual features into the lens or to impart features that affect visual function, for example for the purpose of generating bifocal or multifocal lenses. A preferred class of refractive index moiety are polyamides. Exemplary polyamides include, without limitation, polyvinylpyrrolidone (PVP), polyvinylmethyacetamide (PVMA), polydimethylacrylamide (PDMA), polyvinylacetamide (PNVA), poly(hydroxyethyl(meth)acrylamide), polyacrylamide, and copolymers of two or more thereof.

Some chemical structures of exemplary MAPO-RI, MAPO-LA, BAPO-RI, and BAPO-LA compounds are shown below:

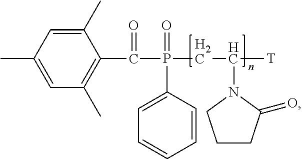

where n is an integer ranging from 10 to 4000, and T is a chain terminating group,

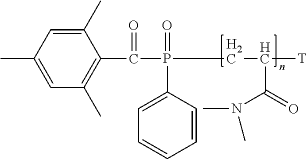

where n is an integer ranging from 10 to 4000, and T is a chain terminating group,

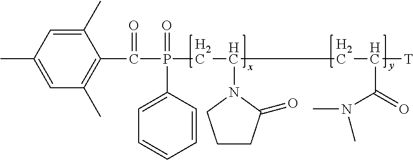

where x and y are independently integers ranging from 10 to 4000, and T is a chain terminating group,

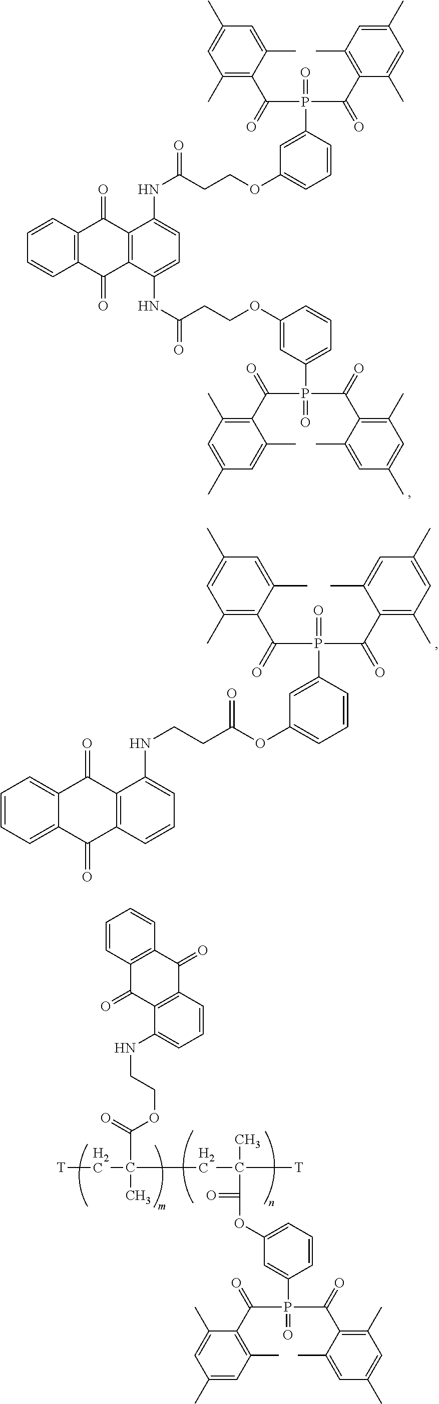

where m is an integer ranging from 10 to 3000 and n is an integer ranging from 1 to 100, and each T is independently a chain terminating group or an initiator fragment,

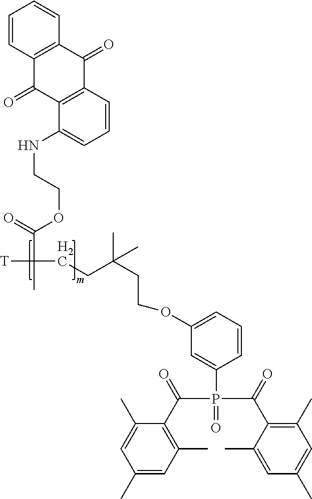

where m is an integer ranging from 10 to 3000, and T is a chain terminating group,

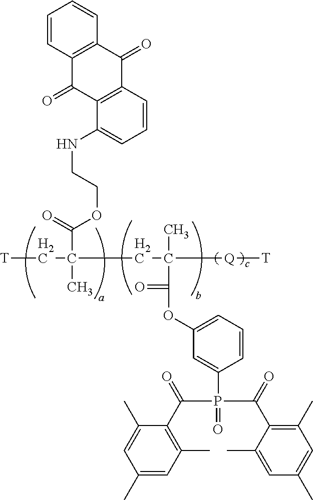

wherein a is an integer ranging from 1 to 100, b is an integer ranging from 1 to 100, c is an integer ranging from 1 to 250, T is independently a chain terminating group or an initiator fragment, and Q is derived from a hydrophilic monomer, such as N,N-dimethylacrylamide, N-vinyl pyrrolidone, 2-hydroxyethyl methacrylate, N-vinyl-N-methylacetamide, N-vinyl acetamide.

The MAPO-RI, MAPO-LA, BAPO-RI, and BAPO-LA compounds may be used in the reactive composition in effective amounts of, for instance, 0.01 weight percent to 20 weight percent based on all components in the reactive monomer mixture, excluding diluents.

According to the invention, after the crosslinked substrate network is formed, portions of the covalently bound activatable free radical initiators are deactivated in one or more selective regions, such that after deactivation, the crosslinked substrate network contains retained covalently bound activatable free radical initiators outside of the one or more selective regions and optionally within the one or more selective regions, depending on the level of deactivation.

Deactivation may be achieved by various means, as long as the covalently bound free radical initiators are converted into other chemical moieties that cannot initiate a free radical polymerization, including but not limited to, oxidation reactions, reduction reactions, and radical coupling reactions. A preferred method of deactivation for crosslinked substate networks containing monoacylphosphine oxides or bisacylphosphine oxides as the covalently bound free radical initiators involves the irradiation of the selective regions in an oxygen gas atmosphere, for example, in air, thereby oxidizing monoacylphosphine oxides or bisacylphosphine oxides into other functional groups.

Ultraviolet or visible light may be used in the deactivation process depending on the composition of the crosslinked substrate network and the desired features imparted by deactivating in the selective regions. The preferred wavelength range of ultraviolet light is between 300 nanometers and 400 nanometers, and the more preferred wavelength range of ultraviolet light is between 350 nanometers and 400 nanometers. The preferred wavelength range of visible light is between 400 nanometers and 500 nanometers, and the more preferred wavelength range of visible light is between 400 nanometers and 450 nanometers. The preferred light source comprises a narrow band light emitting diode.

Deactivation in the crosslinked substrate network may be varied spatially by irradiating in an oxygen atmosphere using a voxel-based lithographic optical forming apparatus, described later in this application, equipped with an actinic radiation light source and a digital micromirror device (DMD) that projects spatially a predetermined DMD script onto the crosslinked substrate network, thereby varying the level of deactivation spatially within the crosslinked substrate network. The DMD script controls the location and the level of deactivation by control the amount of light energy delivered to a specific location within the crosslinked substrate network.

There are several ways in which deactivation may be carried out. for instance, deactivation may be performed as described above on a crosslinked substrate network that enclosed in a mold assembly, comprised of a front mold and a back mold, the front mold and a back mold defining and enclosing a cavity in the shape of the ophthalmic device therebetween.

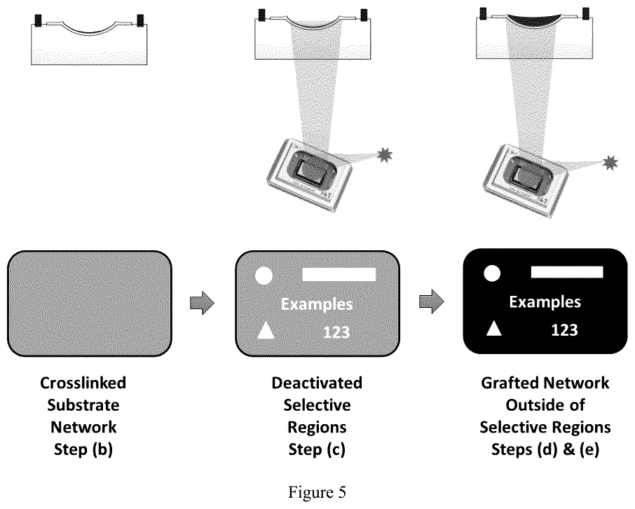

Deactivation may be performed on a crosslinked substrate network that is adhered to either the front or back molds of a mold assembly, preferably adhered to the front mold in a conclave up position as shown in FIG. 5.

Deactivation may be performed on an unextracted, non-hydrated crosslinked substrate network or on an extracted and/or hydrated crosslinked substrate network.

Deactivation may be performed from multiple directions, for example, from the top and from the bottom of a crosslinked substrate network, optionally using more than one actinic radiation light source, more than one DMD, more than one DMD script.

If the crosslinked substrate network comprises an ultraviolet light absorber and if ultraviolet light is used in the deactivation of the selective regions, then the intensity of the ultraviolet light is attenuated inside depending on its path length or depth in the crosslinked substrate network due to absorption. Under these conditions, the deactivation will be more effective on or near the surface of the crosslinked substrate network (due to less absorption), thereby enabling a means to modify the surface or a surface layer by deactivation. In this way, using a two-sided deactivation configuration such as from the top and bottom of the crosslinked substrate network, the retained covalently bound free radical initiators are concentrated in the bulk of the crosslinked substrate network, not in the surface layer, thereby enabling subsequent grafting in only the bulk and preventing grafting on the surface of the grafted polymeric network, or at least substantially so.

If the crosslinked substrate network comprises a visible light absorber and if visible light is used in the deactivation of the selective regions, then the intensity of the visible light may attenuated inside depending on its path length or depth in the crosslinked substrate network due to absorption. Under these conditions, the deactivation may be more effective on or near the surface of the crosslinked substrate network (due to less absorption), thereby enabling a means to modify the surface or a surface layer by deactivation. In this way, using a two-sided deactivation configuration such as from the top and bottom of the crosslinked substrate network, the retained covalently bound free radical initiators may be concentrated in the bulk of the crosslinked substrate network, not in the surface region, thereby enabling subsequent grafting in only the bulk and preventing grafting on the surface of the grafted polymeric network, or at least substantially so.

According to the invention, after deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions, the crosslinked substrate network, formed as described above, is contacted with a grafting composition. The grafting composition contains one or more ethylenically unsaturated compounds. The crosslinked substrate network is a swellable material and therefore absorbs at least some grafting composition for the subsequent grafting reaction. Absorption into the crosslinked substrate network may be carried out in various ways. For instance, the crosslinked substrate network may be placed in the grafting composition and allowed to swell. Or the crosslinked substrate network may be first swollen in a solvent and then combined with the grafting composition, e.g., by suspending the pre-swollen crosslinked substrate network in the grafting composition, during which the reactive components partition into the crosslinked substrate network by molecular diffusion and fluid exchange prior. Alternatively, the crosslinked substrate network may first be extracted with a solvent, either an organic solvent or an aqueous organic solvent or both, optionally followed by hydration and equilibration in aqueous buffers or deionized water, and then combined with the grafting composition as described previously. Any organic solvent or aqueous organic solvent may be used. There is no particular minimum amount of the grafting composition that should absorb into the crosslinked substrate network as long as some is present (greater than 0 weight percent of reactive components). In some embodiments, it may be preferable for the crosslinked substrate network to be swellable in the grafting composition by at least 0.0001 weight percent, alternatively at least 0.01 weight percent, alternatively at least 0.1 weight percent, alternatively at least 5 weight percent, alternatively at least 10 weight percent, or alternatively at least 25 weight percent, at 25° C., relative to its dry weight.

Following the contacting of the crosslinked substrate network with the grafting composition, at least some of the retained activatable free radical initiator of the crosslinked substrate network is activated. For example, if the polymerization initiator used in step (a) of the process is a bisacylphosphine oxide, then retained covalently bound activatable free radical initiators, in this case, monoacylphosphine oxides, outside of the one or more selective regions and optionally within the one or more selective regions, may be activated by irradiation at 420 nanometers or below using an appropriate light source. According to the invention, the activation may be conducted anywhere in the crosslinked substrate network, except of course in selective regions in which all of the monoacylphosphine oxide was deactivated. The grafting composition then undergoes polymerization in the activated portions of the crosslinked substrate network having retained monoacylphosphine oxide. The product is thus an ophthalmic device having localized regions or volumes that comprise a grafted polymeric network and selective regions with either no grafted polymeric network or varying amounts depending on the level of deactivation and therefore the concentration of covalently bound monoacylphosphine oxides. Any un-grafted composition along with any by-products or by-product polymers may be removed from the network, for instance, by extraction with a solvent.

The grafting outside of the selective regions of the crosslinked substrate network, and optionally within the one or more selective regions with partial deactivation, allows a manufacturer to change the chemical composition of the device in those volume elements. As a result, new ophthalmic devices can be prepared that possess desirable mechanical features, physical properties, or optical properties. By way of non-limiting example, in the contact lens field, the inventive process may allow for custom light absorption profiles in the optical zone comprising both clear (deactivated) and colored (grafted to varying degrees) sections or patterns in the optical zone. These custom light absorption profiles may be achieved by completely deactivating some selective regions such as in the center of the optical zone, partially deactivating in other selective regions of the optical zone, and then grafting a colored composition onto all of the regions having retained covalently bound activatable free radical initiators, followed by standard extraction, hydration, and sterilization procedures. Other contact lens examples are the formation of patterns, including cosmetic designs, fiduciary markers, and barcodes in the periphery of the contact lens using similar process, namely create a clear pattern, fiduciary marker, or barcode in the deactivation step and then graft enough of a colored composition to make said patterns, fiduciary markers, or barcodes visible to a third-party observer or optical scanner. The patterns, fiduciary markers, and barcodes can be composed of dots, lines, shapes, symbols, letters, numbers, and the like, with variable sizes and spacing as well as color intensity, thereby enabling high levels of information storage. Such patterns, fiduciary markers, and barcodes can be used in tracking lenses during manufacturing, product surveillance, or clinical trials, can be used as inversion markers to ensure that contact lens wearers place do not invert the lens prior to insertion onto the eye, and can be used as diagnostic tools to better fit a patient with the best prescription lens.

Various techniques may be used to carry out the inventive process comprising the steps: (a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker; (b) subjecting the reactive composition to a first activation step such that the reactive composition polymerizes therein to form a crosslinked substrate network containing a covalently bound activatable free radical initiator; (c) deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions; (d) contacting the crosslinked substrate network with a grafting composition containing one or more ethylenically unsaturated compounds, wherein the contacting is conducted under conditions such that the grafting composition penetrates into the crosslinked substrate network; and (e) activating the retained covalently bound activatable free radical initiator such that the grafting composition polymerizes with the crosslinked substrate network, thereby forming grafted polymeric networks, outside of the selective regions and optionally partially within the selective regions.

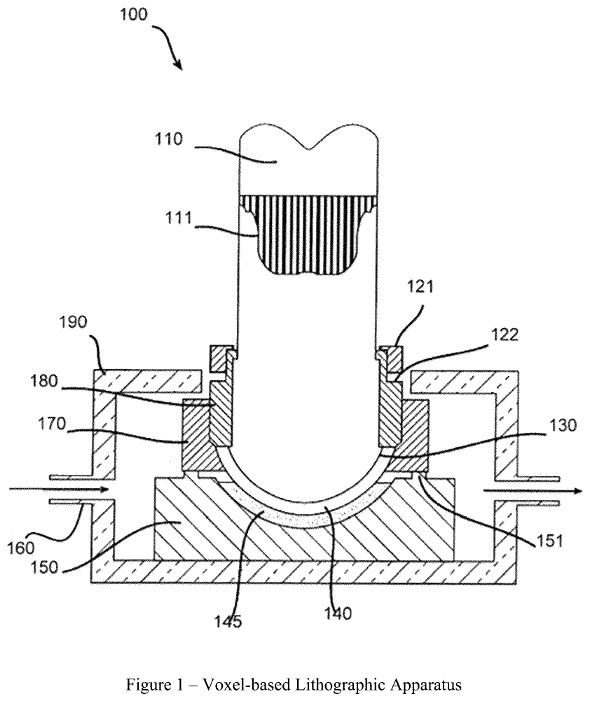

Various techniques may be used for the deactivation of at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions. The same techniques may be used for the activation of the retained covalently bound activatable free radical initiator such that the grafting composition polymerizes with the crosslinked substrate network outside of the deactivated selective regions and optionally partially within the deactivated selective regions. A preferred technique is voxel-based lithography as generally described, for example, in US20150146159, U.S. Pat. Nos. 9,075,186, and 8,317,505, each of which is incorporated herein by reference in its entirety. Additional references include U.S. Pat. Nos. 7,905,594, 8,157,373, 8,240,849, 8,313,828, 8,318,055, 8,795,558, 9,180,633, 9,180,634, 9,417,464, 9,610,742, 9,857,607, U.S. Ser. No. 10/961,341, U.S. Ser. No. 11/021,558, and U.S. Ser. No. 11/034,789, each of which is incorporated herein by reference in its entirety. An exemplary voxel-based lithographic apparatus that may be used in the invention is shown in FIG. 1.

Referring to FIG. 1, the voxel-based lithographic optical forming apparatus 100 may include a source of actinic radiation 110, a spatial grid 111, a forming optic 130 having a forming optic surface 140. In some exemplary embodiments of the forming apparatus 100, the radiation or light may impinge in a roughly vertical manner to the surface 140 of the forming optic 130. The forming optic 130 may be held in place via a retaining ring 121 or other fastening device, which may maintain the correct orientation of the optical system relative to the forming optic 130. Other paths that light may take on a voxel-by-voxel basis across the optic surface 140 may be apparent and are within the scope of the inventive art.

In exemplary embodiments where the relative orientation of a reservoir 150, which may be empty or may contain a liquid 145 such as the grafting composition and forming optic 130 to the light beam may be significant, additional mechanisms for their interlocked location may be included, for example, a forming optic retaining member 170 with associated interlocking features 180 and 122. The alignment between the retaining member 170 and interlocking features 180 and 122 may also provide for position control of the centering of the reservoir 150 to the forming optic surface 140. The position control may be enhanced in some exemplary embodiments with a spacing ring 151, which may also control the volume of material added to the reservoir 150.

The reservoir 150 may be enclosed in a containment vessel 190 that may exclude ambient gasses, such as oxygen. The exclusion may be enhanced by flowing an inert gas, such as nitrogen, through a tube or channel 160 included in the containment vessel 190. In other exemplary embodiments, the oxygen level may be managed by controlling the dilution of oxygen in the gas flowed through the channel 160 included in the containment vessel 190.

The forming optic 130 may be made from numerous optically transparent materials, wherein a light beam, such as actinic radiation, may pass through the forming optic 130 and impinge on the target. For example, the forming optic 130 may comprise fused quartz or transparent polymeric materials.

In some exemplary embodiments, a crosslinked substrate network may first be formed in a mold as describe above. The crosslinked substrate network may then be positioned (not shown) on the forming optic surface 140 and contacted with grafting composition 145 contained in the reservoir 150. Alternatively, a crosslinked substrate network may be formed, for instance in a mold as described above, and contacted with the grafting composition outside of the voxel-based lithograph optical forming apparatus. The crosslinked substrate network, containing grafting composition, may then be positioned (not shown) on the forming optic surface 140. In this embodiment, the reservoir 150 may be empty or it may, for example, contain solvents, water, or additional grafting composition.

Actinic radiation 110, which may be controlled and varied across a spatial grid 111, may be applied to the crosslinked lens substrate (containing grafting composition) at the appropriate wavelength for activating the substrate's covalently bound activatable free radical initiators. The actinic radiation 110 may be controlled such that it impinges at selective locations on the crosslinked substrate network, resulting in localized activation of the covalently bound initiators with consequent grafting at only those locations. Various techniques may be used to control the actinic radiation such that it impinges only at the desired locations of the crosslinked substrate network. For instance, a digital micromirror device (DMD) and DMD script along with various associated components may be used, as described in U.S. Pat. No. 9,075,186. The source of actinic irradiation used in any deactivation step and any activation step (polymerization or grafting) of any of the inventive processes herein may include a plurality of selectively controllable beams of actinic radiation controlled by a digital micro-mirror device according to a predetermined script. The illumination source of the actinic irradiation may include at least one light emitting diode, and the predetermined DMD script may direct the actinic irradiation to one or more surfaces of the crosslinked substrate network.

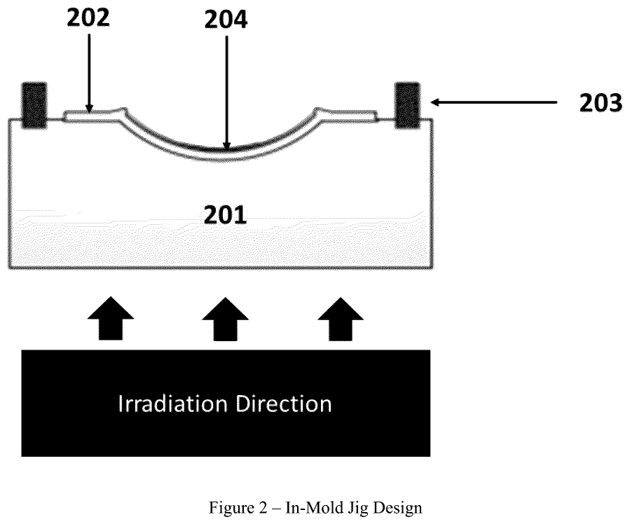

As described previously herein, ultraviolet or visible light may be used in any deactivation step and any activation step (polymerization or grafting) of any of the inventive processes herein. The preferred wavelength range of ultraviolet light is between 300 nanometers and 400 nanometers, and the more preferred wavelength range of ultraviolet light is between 350 nanometers and 400 nanometers. The preferred wavelength range of visible light is between 400 nanometers and 500 nanometers, and the more preferred wavelength range of visible light is between 400 nanometers and 450 nanometers. The preferred light source comprises a narrow band light emitting diode. Although the voxel-based lithograph optical forming apparatus may be configured in different ways, one preferred set-up for making ophthalmic lenses is to use an in-mold jig as shown in FIG. 2. The in-mold jig has a clear front mold base (201) made from a suitable plastic such as poly(methylmethacrylate) that is designed to hold a front mold (202) with a crosslinked substrate network lens (204) adhered thereon in a concave up position, thereby allowing the grafting composition to be dispensed therein. The front mold base is also designed to be attached to the voxel-based lithograph optical forming apparatus for actinic irradiation using a predetermined DMD script. The targeted image plane of the apparatus may be adjusted to be at or near the center of the front mold (202). The in-mold jig is further equipped with magnetic spacers that hold the front mold in place. These magnetic spacers may also be designed to hold a complete contact lens mold assembly, comprised of a front mold and a back mold, the front mold and a back mold defining and enclosing a cavity in the shape of the contact lens therebetween, as well as an optional (not shown in FIG. 2) clear back mold base having an opaque or light absorbing top surface to control the amount of extraneous light. The ladder configuration provides for the mold assembly having a precursor crosslinked substrate network lens, comprising MAPO-M, MAPO-RI, MAPO-LA, BAPO-M, BAPO-RI, and BAPO-LA compounds, to be irradiated conveniently in preselected regions prior to completing the thermal polymerization, thereby forming a crosslinked substrate network lens ready for grafting.

While voxel-based lithography as described above is a preferred technique for the selective activation of the crosslinked substrate network, other techniques may also be utilized. For example, selective activation may be provided by simply masking, from the activating light, those areas of the crosslinked substrate network where activation is not desired. The non-masked areas of the substrate may then undergo activation and grafting. Unreacted material may be removed, for instance, by extraction.

Additional optional grafting steps may be added. Such additional grafting may, for instance, be through the bulk of the grafted crosslinked substrate network, or it may be more concentrated at the surface than at the core, or it may be localized in other regions of the grafted crosslinked substrate network. For instance, following the above-described localized grafting, the grafted crosslinked substrate network may be contacted with a second grafting composition containing one or more ethylenically unsaturated compounds. Such second composition may be grafted onto the grafted crosslinked substrate network if the grafted crosslinked substrate network contains additional retained covalently bound activatable free radical initiators.

It should be noted that the free radical initiator covalently bound to the crosslinked substrate network forms two free radical groups when activated, one of which may not be covalently bound to the substrate. Consequently, some of the reactive components in the grafting composition may polymerize via the unbound free radical group to form a polymer that is not covalently bound with the network. Such polymer is referred to herein as a “byproduct polymer.” This byproduct polymer may be induced to covalently bind with the grafted polymeric network by inclusion of a crosslinking agent in the grafting composition. The composition may contain at least a portion of the byproduct polymer that is not covalently bound to the grafted polymeric network. To achieve this, the polymerization of the grafting composition is conducted in the substantial absence of a crosslinker. By “substantial absence of a crosslinker” is meant that any crosslinker used in the grafting composition is present in less than a stoichiometric amount. In some embodiments, no crosslinker is present in the grafting composition.

The reactive composition and the grafting compositions of the invention contain ethylenically unsaturated compounds as reactive components. The ethylenically unsaturated compounds undergo polymerization to form the polymer compositions described herein. As will be appreciated, a wide variety of ethylenically unsaturated compounds may be used in the invention.

The ethylenically unsaturated compounds may be the same or different between the reactive composition and the grafting composition, although in some embodiments, it is preferable that at least some of the ethylenically unsaturated compounds in each composition are different. By using materials for the reactive composition that are different from the grafting composition, it becomes possible to design ophthalmic devices that combine desirable properties from materials that may otherwise not be readily compatible. This is one of the advantages of the invention.

The ethylenically unsaturated compound for inclusion in the reactive composition and/or the grafting composition may comprise an independently selected silicone-containing component.

The silicone-containing component may comprise one or more compounds selected from monomers or macromer, where each compound may independently comprise at least one polymerizable group, at least one siloxane group, and one or more linking groups connecting the polymerizable group(s) to the siloxane group(s). The silicone-containing components may, for instance, contain from 1 to 220 siloxane repeat units, such as the groups defined below. The silicone-containing component may also contain at least one fluorine atom.

The silicone-containing component may comprise: one or more polymerizable groups as defined above; one or more optionally repeating siloxane units; and one or more linking groups connecting the polymerizable groups to the siloxane units. The silicone-containing component may comprise: one or more polymerizable groups that are independently a (meth)acrylate, a styryl, a vinyl ether, a (meth)acrylamide, an N-vinyllactam, an N-vinylamide, an O-vinylcarbamate, an O-vinylcarbonate, a vinyl group, or mixtures of the foregoing; one or more optionally repeating siloxane units; and one or more linking groups connecting the polymerizable groups to the siloxane units.

The silicone-containing component may comprise: one or more polymerizable groups that are independently a (meth)acrylate, a (meth)acrylamide, an N-vinyl lactam, an N-vinylamide, a styryl, or mixtures of the foregoing; one or more optionally repeating siloxane units; and one or more linking groups connecting the polymerizable groups to the siloxane units.

The silicone-containing component may comprise: one or more polymerizable groups that are independently a (meth)acrylate, a (meth)acrylamide, or mixtures of the foregoing; one or more optionally repeating siloxane units; and one or more linking groups connecting the polymerizable groups to the siloxane units.

The silicone-containing component may comprise one or more polymerizable compounds of Formula A:

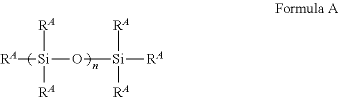

wherein:
at least one R^{A} is a group of formula R_{g}-L- wherein R_{g} is a polymerizable group and L is a linking group, and the remaining R^{A} are each independently:
   (a) R_{g}-L-,
   (b) C₁-C₁₆ alkyl optionally substituted with one or more hydroxy, amino, amido, oxa, carboxy, alkyl carboxy, carbonyl, alkoxy, amido, carbamate, carbonate, halo, phenyl, benzyl, or combinations thereof,
   (c) C₃-C₁₂ cycloalkyl optionally substituted with one or more alkyl, hydroxy, amino, amido, oxa, carbonyl, alkoxy, amido, carbamate, carbonate, halo, phenyl, benzyl, or combinations thereof,
   (d) a C₆-C₁₄ aryl group optionally substituted with one or more alkyl, hydroxy, amino, amido, oxa, carboxy, alkyl carboxy, carbonyl, alkoxy, amido, carbamate, carbonate, halo, phenyl, benzyl, or combinations thereof,
   (e) halo,
   (f) alkoxy, cyclic alkoxy, or aryloxy,
   (g) siloxy,
   (h) alkyleneoxy-alkyl or alkoxy-alkyleneoxy-alkyl, such as polyethyleneoxyalkyl, polypropyleneoxyalkyl, or poly(ethyleneoxy-co-propyleneoxyalkyl), or
   (i) a monovalent siloxane chain comprising from 1 to 100 siloxane repeat units optionally substituted with alkyl, alkoxy, hydroxy, amino, oxa, carboxy, alkyl carboxy, alkoxy, amido, carbamate, halo or combinations thereof; and
n is from 0 to 500 or from 0 to 200, or from 0 to 100, or from 0 to 20, where it is understood that when n is other than 0, n is a distribution having a mode equal to a stated value. When n is 2 or more, the SiO units may carry the same or different R^{A} substituents and if different R^{A} substituents are present, the n groups may be in random or block configuration.

In Formula A, three R^{A} may each comprise a polymerizable group, alternatively two R^{A} may each comprise a polymerizable group, or alternatively one R^{A} may comprise a polymerizable group.

Examples of silicone-containing components suitable for use in the invention include, but are not limited to, compounds listed in Table B. Where the compounds in Table B contain polysiloxane groups, the number of SiO repeat units in such compounds, unless otherwise indicated, is preferably from 3 to 100, more preferably from 3 to 40, or still more preferably from 3 to 20.

TABLE B

| | |
|---|---|
| 1 | mono-methacryloxypropyl terminated mono-n-butyl terminated polydimethylsiloxanes |
| | (mPDMS) (preferably containing from 3 to 15 SiO repeating units) |
| 2 | mono-acryloxypropyl terminated mono-n-butyl terminated polydimethylsiloxane |
| 3 | mono(meth)acryloxypropyl terminated mono-n-methyl terminated |
| | polydimethylsiloxane |
| 4 | mono(meth)acryloxypropyl terminated mono-n-butyl terminated polydiethylsiloxane |
| 5 | mono(meth)acryloxypropyl terminated mono-n-methyl terminated polydiethylsiloxane |
| 6 | mono(meth)acrylamidoalkylpolydialkylsiloxanes |
| 7 | mono(meth)acryloxyalkyl terminated mono-alkyl polydiarylsiloxanes |
| 8 | 3-methacryloxypropyltris(trimethylsiloxy)silane (TRIS) |
| 9 | 3-methacryloxypropylbis(trimethylsiloxy)methylsilane |
| 10 | 3-methacryloxypropylpentamethyl disiloxane |
| 11 | mono(meth)acrylamidoalkylpolydialkylsiloxanes |
| 12 | mono(meth)acrylamidoalkyl polydimethylsiloxanes |
| 13 | N-(2,3-dihydroxypropane)-N'-(propyl tetra(dimethylsiloxy) |
| | dimethylbutylsilane)acrylamide |
| 14 | N-[3-tris(trimethylsiloxy)silyl]-propyl acrylamide (TRIS-Am) |
| 15 | 2-hydroxy-3-[3-methyl-3,3-di(trimethylsiloxy)silylpropoxy]-propyl methacrylate |
| | (SIMAA) |
| 16 | 2-hydroxy-3-methacryloxypropyloxypropyl-tris(trimethylsiloxy)silane |
| 17 | 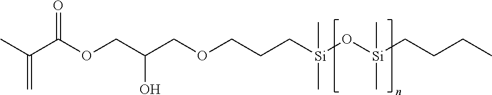 |
| | mono-(2-hydroxy-3-methacryloxypropyloxy)-propyl terminated mono-n-butyl |
| | terminated polydimethylsiloxanes (OH-mPDMS) (containing from 4 to 30, or from 4 to |
| | 20, or from 4 to 15 SiO repeat units) |
| 18 | 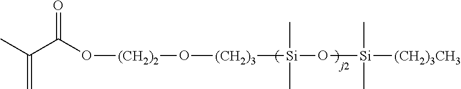 |
| 19 | 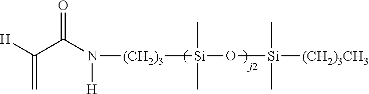 |
| 20 | 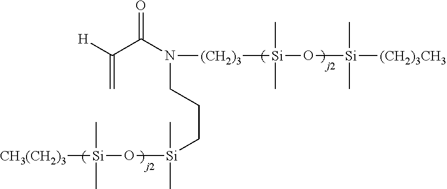 |
| 21 | 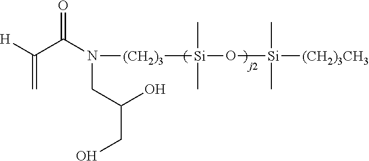 |
| 22 | 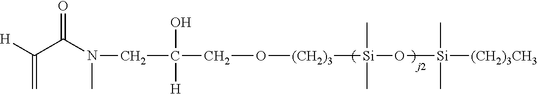 |
| 23 | 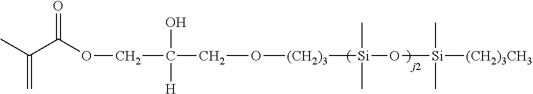 |
| 24 | 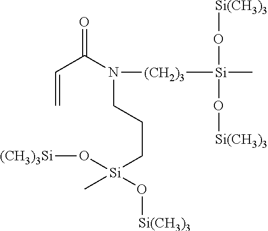 |

Additional non-limiting examples of suitable silicone-containing components are listed in Table C. Unless otherwise indicated, j2 where applicable is preferably from 1 to 100, more preferably from 3 to 40, or still more preferably from 3 to 15. In compounds containing j1 and j2, the sum of j1 and j2 is preferably from 2 to 100, more preferably from 3 to 40, or still more preferably from 3 to 15.

TABLE C

| | |
|---|---|
| 25 | 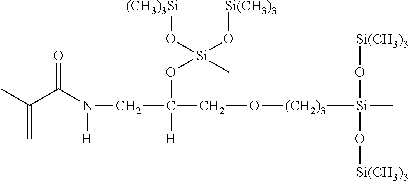 |
| 26 | 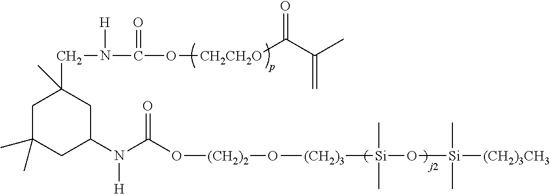 p is 1 to 10 |
| 27 | 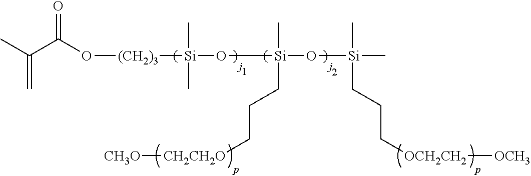 p is 5-10 |
| 28 | 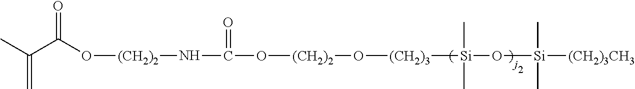 |
| 29 | 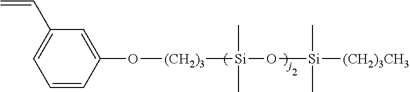 |
| 30 | 1,3-bis[4-(vinyloxycarbonyloxy)but-1-yl]tetramethyl-disiloxane |
| 31 | 3-(vinyloxycarbonylthio) propyl-[tris (trimethylsiloxy)silane] |
| 32 | 3-[tris(trimethylsiloxy)silyl] propyl allyl carbamate |
| 33 | 3-[tris(trimethylsiloxy)silyl] propyl vinyl carbamate |
| 34 | tris(trimethylsiloxy)silylstyrene (Styryl-TRIS) |
| 35 | 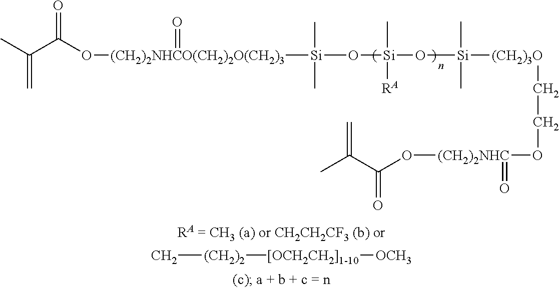 |
| 36 | 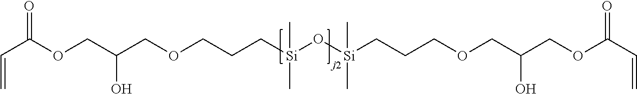 |
| 37 | 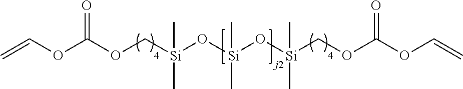 |
| 38 | 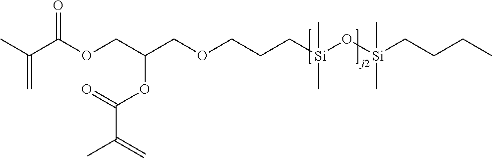 |
| 39 | 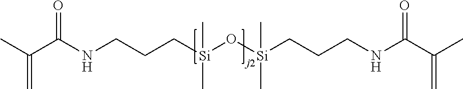 |
| 40 | 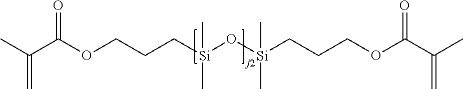 |
| 41 | 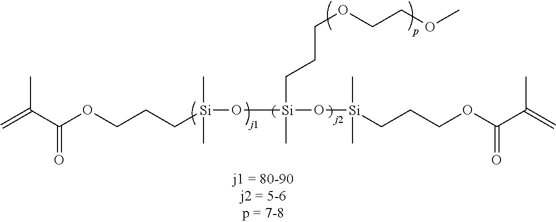 |
| 42 | 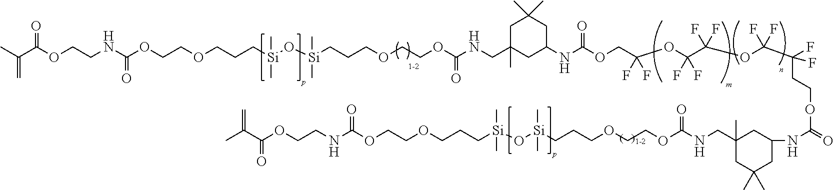 m ≈ 3.5-5.5; n ≈ 4-6.5; p ≈ 22-26 |
| 43 | 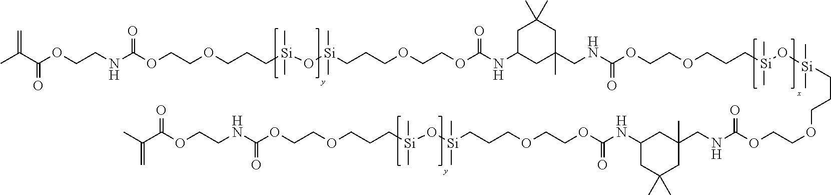 IEM-PDMS(Mn ≈ 3000)-IPDI-PDMS(Mn ≈ 2000)-IPDI-PDMS(Mn ≈ 3000)-IEM (see WO2016100457) |

Mixtures of silicone-containing components may be used. By way of example, suitable mixtures may include, but are not limited to: a mixture of mono-(2-hydroxy-3-methacryloxypropyloxy)-propyl terminated mono-n-butyl terminated polydimethylsiloxane (OH-mPDMS) having different molecular weights, such as a mixture of OH-mPDMS containing 4 and 15 SiO repeat units; a mixture of OH-mPDMS with different molecular weights (e.g., containing 4 and 15 repeat SiO repeat units) together with a silicone based crosslinker, such as bis-3-acryloxy-2-hydroxypropyloxypropyl polydimethylsiloxane (ac-PDMS); a mixture of 2-hydroxy-3-[3-methyl-3,3-di(trimethylsiloxy)silylpropoxy]-propyl methacrylate (SiMAA) and mono-methacryloxypropyl terminated mono-n-butyl terminated polydimethylsiloxane (mPDMS), such as mPDMS 1000.

Silicone-containing components for use in the invention may have an average molecular weight of from about 400 to about 4000 daltons.

The silicone containing component(s) may be present in amounts up to about 95 weight %, or from about 10 to about 80 weight %, or from about 20 to about 70 weight %, based upon all reactive components of a formulation (excluding diluents).

The ethylenically unsaturated compound for inclusion in the reactive composition and/or the grafting composition may comprise an independently selected hydrophilic component. Hydrophilic components include those which are capable of providing at least about 20% or at least about 25% water content to the resulting composition when combined with the remaining reactive components. Suitable hydrophilic components include hydrophilic monomers, prepolymers and polymers. Preferably, the hydrophilic component has at least one polymerizable group and at least one hydrophilic functional group. Examples of polymerizable groups include acrylic, methacrylic, acrylamido, methacrylamido, fumaric, maleic, styryl, isopropenylphenyl, O-vinylcarbonate, O-vinylcarbamate, allylic, O-vinylacetyl and N-vinyllactam and N-vinylamido double bonds.

The term “vinyl-type” or “vinyl-containing” monomers refer to monomers containing the vinyl grouping (—CH═CH₂) and are generally highly reactive. Such hydrophilic vinyl-containing monomers are known to polymerize relatively easily.

“Acrylic-type” or “acrylic-containing” monomers are those monomers containing an acrylic group (CH₂═CRCOX) wherein R is H or CH₃, and X is O or N, which are also known to polymerize readily, such as N,N-dimethyl acrylamide (DMA), 2-hydroxyethyl methacrylamide, polyethyleneglycol monomethacrylate, methacrylic acid, acrylic acid, mixtures thereof and the like.

Hydrophilic monomers with at least one hydroxyl group (hydroxyalkyl monomer) may be used. The hydroxyl alkyl group may be selected from C₂-C₄ mono or dihydroxy substituted alkyl, and poly(ethylene glycol) having 1-10 repeating units; or is selected from 2-hydroxyethyl, 2,3-dihydroxypropyl, or 2-hydroxypropyl, and combinations thereof.

Examples of hydroxyalkyl monomers include 2-hydroxyethyl (meth)acrylate, 3-hydroxypropyl (meth)acrylate, 2-hydroxypropyl (meth)acrylate, 2,3-dihydroxypropyl (meth)acrylate, 2-hydroxybutyl (meth)acrylate, 3-hydroxybutyl (meth)acrylate, 1-hydroxypropyl 2-(meth)acrylate, 2-hydroxy-2-methyl-propyl (meth)acrylate, 3-hydroxy-2,2-dimethyl-propyl (meth)acrylate, 4-hydroxybutyl (meth)acrylate, 2-hydroxyethyl (meth)acrylamide, N-(2-hydroxypropyl) (meth)acrylamide, N,N-bis(2-hydroxyethyl) (meth)acrylamide, N,N-bis(2-hydroxypropyl) (meth)acrylamide, N-(3-hydroxypropyl) (meth)acrylamide, 2,3-dihydroxypropyl (meth)acrylamide, glycerol (meth)acrylate, polyethyleneglycol monomethacrylate, and mixtures thereof.

The hydroxyalkyl monomer may also be selected from the group consisting of 2-hydroxyethyl methacrylate, glycerol methacrylate, 2-hydroxypropyl methacrylate, hydroxybutyl methacrylate, 3-hydroxy-2,2-dimethyl-propyl methacrylate, and mixtures thereof.

The hydroxyalkyl monomer may comprise 2-hydroxyethyl methacrylate, 3-hydroxy-2,2-dimethyl-propyl methacrylate, hydroxybutyl methacrylate or glycerol methacrylate.

When hydrophilic polymers in quantities great than about 3 wt % are desired, hydroxyl containing (meth)acrylamides are generally too hydrophilic to be included as compatibilizing hydroxyalkyl monomers, and hydroxyl containing (meth)acrylates may be included in the reactive composition and the lower amount of hydroxyalkyl monomers may be selected to provide a haze value to the final lens of less than about 50% or less than about 30%.

It will be appreciated that the amount of hydroxyl component will vary depending upon a number of factors, including, the number of hydroxyl groups on the hydroxyalkyl monomer, the amount, molecular weight and presence of hydrophilic functionality on the silicone containing components. The hydrophilic hydroxyl component may be present in the reactive composition in amounts up to about 15%, up to about 10 wt %, between about 3 and about 15 wt % or about 5 and about 15 wt %.

Hydrophilic vinyl-containing monomers which may be incorporated into the polymer compositions include monomers such as hydrophilic N-vinyl lactam and N-vinyl amide monomers including: N-vinyl pyrrolidone (NVP), N-vinyl-2-piperidone, N-vinyl-2-caprolactam, N-vinyl-3-methyl-2-caprolactam, N-vinyl-3-methyl-2-piperidone, N-vinyl-4-methyl-2-piperidone, N-vinyl-4-methyl-2-caprolactam, N-vinyl-3-ethyl-2-pyrrolidone, N-vinyl-4,5-dimethyl-2-pyrrolidone, N-vinyl acetamide (NVA), N-vinyl-N-methylacetamide (VMA), N-vinyl-N-ethyl acetamide, N-vinyl-N-ethyl formamide, N-vinyl formamide, N-vinyl-N-methylpropionamide, N-vinyl-N-methyl-2-methylpropionamide, N-vinyl-2-methylpropionamide, N-vinyl-N,N′-dimethylurea, 1-methyl-3-methylene-2-pyrrolidone, 1-methyl-5-methylene-2-pyrrolidone, 5-methyl-3-methylene-2-pyrrolidone; 1-ethyl-5-methylene-2-pyrrolidone, N-methyl-3-methylene-2-pyrrolidone, 5-ethyl-3-methylene-2-pyrrolidone, 1-N-propyl-3-methylene-2-pyrrolidone, 1-N-propyl-5-methylene-2-pyrrolidone, 1-isopropyl-3-methylene-2-pyrrolidone, 1-isopropyl-5-methylene-2-pyrrolidone, N-vinyl-N-ethyl acetamide, N-vinyl-N-ethyl formamide, N-vinyl formamide, N-vinyl isopropylamide, N-vinyl caprolactam, N-carboxyvinyl-β-alanine (VINAL), N-carboxyvinyl-α-alanine, N-vinylimidazole, and mixtures thereof.

Hydrophilic O-vinyl carbamates and O-vinyl carbonates monomers that may be used in the invention include: N-2-hydroxyethyl vinyl carbamate and N-carboxy-ß-alanine N-vinyl ester. Further examples of the hydrophilic vinyl carbonate or vinyl carbamate monomers are disclosed in U.S. Pat. No. 5,070,215, and the hydrophilic oxazolone monomers are disclosed in U.S. Pat. No. 4,910,277.

Examples of vinyl carbamates and carbonates that may be used include: N-2-hydroxyethyl vinyl carbamate, N-carboxy-ß-alanine N-vinyl ester, other hydrophilic vinyl monomers, including vinylimidazole, ethylene glycol vinyl ether (EGVE), di(ethylene glycol) vinyl ether (DEGVE), allyl alcohol, 2-ethyl oxazoline, vinyl acetate, acrylonitrile, and mixtures thereof.

(Meth)acrylamide monomers may also be used as hydrophilic monomers. Examples include N—N-dimethylacrylamide, acrylamide, N,N-bis(2-hydroxyethyl)acrylamide, acrylonitrile, N-isopropyl acrylamide, N,N-dimethylaminopropyl(meth)acrylamide, and any of the hydroxyl functional (meth)acrylamides listed above.

The hydrophilic monomers which may be incorporated into the polymers disclosed herein may be selected from N,N-dimethyl acrylamide (DMA), 2-hydroxyethyl acrylamide, 2-hydroxyethyl methacrylamide, N-hydroxypropyl methacrylamide, bishydroxyethyl acrylamide, 2,3-dihydroxypropyl (meth)acrylamide, N-vinylpyrrolidone (NVP), N-vinyl-N-methyl acetamide, N-vinyl methacetamide (VMA), and polyethyleneglycol monomethacrylate.

The hydrophilic monomers may be selected from DMA, NVP, VMA, NVA, and mixtures thereof.

The hydrophilic monomers may be macromers of linear or branched poly(ethylene glycol), poly(propylene glycol), or statistically random or block copolymers of ethylene oxide and propylene oxide. The macromer of these polyethers has one polymerizable group. Non-limiting examples of such polymerizable groups are acrylates, methacrylates, styrenes, vinyl ethers, acrylamides, methacrylamides, and other vinyl compounds. The macromer of these polyethers may comprise acrylates, methacrylates, acrylamides, methacrylamides, and mixtures thereof. Other suitable hydrophilic monomers will be apparent to one skilled in the art.

The hydrophilic monomers may also comprise charged monomers including but not limited to acrylic acid, methacrylic acid, 3-acrylamidopropionic acid (ACA1), 4-acrylamidobutanoic acid, 5-acrylamidopentanoic acid (ACA2), 3-acrylamido-3-methylbutanoic acid (AMBA), N-vinyloxycarbonyl-α-alanine, N-vinyloxycarbonyl-β-alanine (VINAL), 2-vinyl-4,4-dimethyl-2-oxazolin-5-one (VDMO), reactive sulfonate salts, including, sodium-2-(acrylamido)-2-methylpropane sulphonate (AMPS), 3-sulphopropyl (meth)acrylate potassium salt, 3-sulphopropyl (meth)acrylate sodium salt, bis 3-sulphopropyl itaconate di sodium, bis 3-sulphopropyl itaconate di potassium, vinyl sulphonate sodium salt, vinyl sulphonate salt, styrene sulfonate, sulfoethyl methacrylate, combinations thereof and the like.

The hydrophilic monomers may be selected from N, N-dimethyl acrylamide (DMA), N-vinylpyrrolidone (NVP), 2-hydroxyethyl methacrylate (HEMA), N-vinyl methacetamide (VMA), and N-vinyl N-methyl acetamide (NVA), N-hydroxypropyl methacrylamide, mono-glycerol methacrylate, 2-hydroxyethyl acrylamide, 2-hydroxyethyl methacrylamide, bishydroxyethyl acrylamide, 2,3-dihydroxypropyl (meth)acrylamide and mixtures thereof.

The hydrophilic monomers may be selected from DMA, NVP, HEMA, VMA, NVA, and mixtures thereof.

The hydrophilic monomer(s) (including the hydroxyl alkyl monomers) may be present in amounts up to about 60 wt %, from about 1 to about 60 weight %, from about 5 to about 50 weight %, or from about 5 to about 40 weight %, based upon the weight of all reactive components.

Other hydrophilic monomers that can be employed include polyoxyethylene polyols having one or more of the terminal hydroxyl groups replaced with a polymerizable group. Examples include polyethylene glycol with one or more of the terminal hydroxyl groups replaced with a polymerizable group. Examples include polyethylene glycol reacted with one or more molar equivalents of an end-capping group such as isocyanatoethyl methacrylate (“IEM”), methacrylic anhydride, methacryloyl chloride, vinylbenzoyl chloride, or the like, to produce a polyethylene polyol having one or more terminal polymerizable olefinic groups bonded to the polyethylene polyol through linking moieties such as carbamate or ester groups.

Still further examples are the hydrophilic vinyl carbonate or vinyl carbamate monomers disclosed in U.S. Pat. No. 5,070,215, and the hydrophilic oxazolone monomers disclosed in U.S. Pat. No. 4,190,277. Other suitable hydrophilic monomers will be apparent to one skilled in the art.

Hydrophilic monomers which may be incorporated into the polymer compositions disclosed herein include hydrophilic monomers such as N,N-dimethyl acrylamide (DMA), 2-hydroxyethyl acrylate, glycerol methacrylate, 2-hydroxyethyl methacrylamide, N-vinylpyrrolidone (NVP), N-vinyl methacrylamide, HEMA, and poly(ethyleneglycol) methyl ether methacrylate (mPEG).

Hydrophilic monomers may include DMA, NVP, HEMA and mixtures thereof.

The reactive composition and/or the grafting composition may contain one or more independently selected ethylenically unsaturated zwitterionic compounds, such as an ethylenically unsaturated betaine. Preferably, the zwitterionic compound is in the grafting composition. Examples of suitable compounds include: N-(2-carboxyethyl)-N,N-dimethyl-3-[(1-oxo-2-propen-1-yl)amino]-1-propanaminium, inner salt (CAS 79704-35-1, also known as 3-acrylamido-N-(2-carboxyethyl)-N,N-dimethylpropane-1-aminium or CBT); 3-methacrylamido-N-(2-carboxyethyl)-N,N-dimethylpropane-1-aminium; N,N-dimethyl-N-[3-[(1-oxo-2-propen-1-yl)amino]propyl]-3-sulfo-1-propanaminium, inner salt (CAS 80293-60-3, also known as 3-((3-acrylamidopropyl) dimethylammonio) propane-1-sulfonate or SBT); 3-((3-methacrylamidopropyl) dimethylammonio) propane-1-sulfonate; 3,5-Dioxa-8-aza-4-phosphaundec-10-en-1-aminium, 4-hydroxy-N,N,N-trimethyl-9-oxo, inner salt, 4-oxide (CAS 163674-35-9, “PBT”); 2-(acrylamidoethoxy)-(2-(trimethylammonio)ethyl) phosphate; 2-(methacrylamidoethoxy)-(2-(trimethylammonio)ethyl) phosphate; 4-hydroxy-N,N,N,10-tetramethyl-9-oxo-3,5,8-trioxa-4-phosphaundec-10-en-1-aminium inner salt, 4-oxide (CAS 67881-98-5, also known as 2-(methacryloyloxy)ethyl (2-(trimethylammonio)ethyl) phosphate or MPC); or 2-(acryloyloxy)ethyl (2-(trimethylammonio)ethyl) phosphate.

The reactive composition and/or the grafting composition may contain one or more independently selected ethylenically unsaturated quaternary ammonium salts. Preferably, the quaternary ammonium salt is in the grafting composition. Examples of suitable compounds include 2-(methacryloyloxy)ethyl trimethylammonium chloride; 2-(acryloyloxy)ethyl trimethylammonium chloride; 3-methacrylamido-N,N,N-trimethylpropan-1-aminium chloride; or 3-acrylamido-N,N,N-trimethylpropan-1-aminium chloride

The reactive composition and/or the grafting composition may contain one or more independently selected ethylenically unsaturated active pharmaceutical ingredients. Preferably, the active pharmaceutical compound is in the grafting composition. Examples of suitable compounds include cyclosporine or salicylate monomers.

The reactive composition and/or the grafting composition may contain one or more independently selected ethylenically unsaturated peptides. Preferably, the peptide is in the grafting composition. Exemplary compounds include, for instance, those wherein the amino-terminus of a peptide may be acylated with an acylating agent such as (meth)acryloyl chloride, (meth)acrylic anhydride, isopropenyl α,α-dimethylbenzyl isocyanate and 2-isocyanatoethyl methacrylate along with known co-reagents and catalysts to form a monomer suitable for incorporation into reactive compositions of the present inventions

The reactive composition of the invention contains a crosslinker. Crosslinkers may optionally be present in the grafting composition. A variety of crosslinkers may be used, including silicone-containing and non-silicone containing cross-linking agents, and mixtures thereof. Examples of suitable crosslinkers include ethylene glycol dimethacrylate (EGDMA), diethyleneglycol dimethacrylate, trimethylolpropane trimethacrylate (TMPTMA), tetraethylene glycol dimethacrylate (TEGDMA), triallyl cyanurate (TAC), glycerol trimethacrylate, 1,3-propanediol dimethacrylate; 2,3-propanediol dimethacrylate; 1,6-hexanediol dimethacrylate; 1,4-butanediol dimethacrylate, methacryloxyethyl vinylcarbonate (HEMAVc), allylmethacrylate, methylene bisacrylamide (MBA), polyethylene glycol dimethacrylate (wherein the polyethylene glycol preferably has a molecular weight up to 5,000 Daltons). The crosslinkers are used in the typical amounts known to those skilled in the art, e.g., from about 0.000415 to about 0.0156 mole per 100 grams of reactive components in the reaction composition.

It should be noted that if the ethylenically unsaturated compound, such as a hydrophilic monomer or a silicone containing monomer, acts as the crosslinker, for instance by virtue of being bifunctional or multifunctional, the addition of a separate crosslinker to the reaction composition is optional. In this case, the ethylenically unsaturated compound is also considered a crosslinker. Examples of hydrophilic monomers which can act as the crosslinking agent and when present do not require the addition of an additional crosslinking agent to the reaction composition include polyoxyethylene polyols described above containing two or more terminal methacrylate moieties. An example of a silicone containing monomer which can act as a crosslinking agent and, when present, does not require the addition of a crosslinking monomer to the reaction composition includes α,ω-bismethacryloypropyl polydimethylsiloxane. In addition, any of the above disclosed multifunctional silicone-containing components may be used as cross-linking agents.

Either or both of the reactive composition and the grafting composition may contain additional components such as, but not limited to, UV absorbers, photochromic compounds, pharmaceutical and nutraceutical compounds, antimicrobial compounds, reactive tints, pigments, copolymerizable and non-polymerizable dyes, release agents and combinations thereof. Other components that can be present in the first and/or grafting compositions include wetting agents, such as those disclosed in U.S. Pat. No. 6,367,929, WO03/22321, WO03/22322, compatibilizing components, such as those disclosed in US2003/162862 and US2003/125498. A preferred UV absorber is 2-(2′-hydroxy-5-methacrylyloxyethylphenyl)-2H-benzotriazole, commonly known as Norbloc. The sum of additional components may be up to about 20 wt %. The reactive compositions may comprise up to about 18 wt % wetting agent, or from about 5 and about 18 wt % wetting agent.

As used herein, wetting agents are hydrophilic polymers having a weight average molecular weight greater than about 5,000 Daltons, between about 150,000 Daltons to about 2,000,000 Daltons; between about 300,000 Daltons to about 1,800,000 Daltons; or between about 500,000 Daltons to about 1,500,000 Daltons.

The amount of optional wetting agent which may be added to the reactive composition and/or the grafting composition of the present invention may be varied depending on the other components used and the desired properties of the resulting product. When present, the internal wetting agents in reactive compositions may be included in amounts from about 1 weight percent to about 20 weight percent; from about 2 weight percent to about 15 percent, or from about 2 to about 12 percent, all based upon the total weight of all of the reactive components. Preferably, a wetting agent, when used, is present in the reactive composition.

Wetting agents include but are not limited to homopolymers, statistically random copolymers, diblock copolymers, triblock copolymers, segmented block copolymers, graft copolymers, and mixtures thereof. Non-limiting examples of internal wetting agents are polyamides, polyesters, polylactones, polyimides, polylactams, polyethers, polyacids homopolymers and copolymers prepared by the free radical polymerization of suitable monomers including acrylates, methacrylates, styrenes, vinyl ethers, acrylamides, methacrylamides, N-vinyllactams, N-vinylamides, O-vinylcarbamates, O-vinylcarbonates, and other vinyl compounds. The wetting agents may be made from any hydrophilic monomer, including those listed herein.

The wetting agents may include acyclic polyamides that comprise pendant acyclic amide groups and are capable of association with hydroxyl groups. Cyclic polyamides comprise cyclic amide groups and are also capable of association with hydroxyl groups.

Examples of suitable acyclic polyamides include polymers and copolymers comprising repeating units of Formula XXIX or Formula XXX:

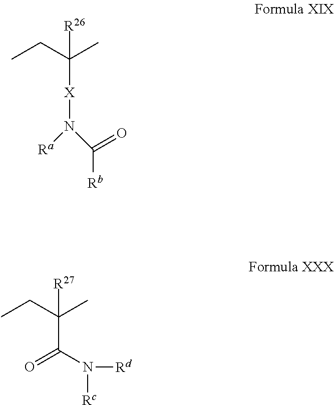

wherein X is a direct bond, —(CO)—, or —(CO)—NHR^{e}—, wherein R²⁶ and R²⁷ are H or methyl groups; wherein R^{e} is a C₁ to C₃ alkyl group; R^{a} is selected from H, straight or branched, substituted or unsubstituted C₁ to C₄ alkyl groups; R^{b} is selected from H, straight or branched, substituted or unsubstituted C₁ to C₄ alkyl groups, amino groups having up to two carbon atoms, amide groups having up to four carbon atoms, and alkoxy groups having up to two carbon groups; R^{c} is selected from H, straight or branched, substituted or unsubstituted C₁ to C₄ alkyl groups, or methyl, ethoxy, hydroxyethyl, and hydroxymethyl; R^{d} is selected from H, straight or branched, substituted or unsubstituted C₁ to C₄ alkyl groups; or methyl, ethoxy, hydroxyethyl, and hydroxymethyl wherein the number of carbon atoms in R^{a} and R^{b} taken together is 8 or less, including 7, 6, 5, 4, 3, or less, and wherein the number of carbon atoms in R^{c} and R^{d} taken together is 8 or less, including 7, 6, 5, 4, 3, or less. The number of carbon atoms in R^{a} and R^{b} taken together may be 6 or less or 4 or less. The number of carbon atoms in R^{c} and R^{d} taken together may be 6 or less. As used herein substituted alkyl groups include alkyl groups substituted with an amine, amide, ether, hydroxyl, carbonyl, carboxy groups or combinations thereof.

R^{a} and R^{b} can be independently selected from H, substituted or unsubstituted C₁ to C₂ alkyl groups. X may be a direct bond, and R^{a} and R^{b} may be independently selected from H, substituted or unsubstituted C₁ to C₂ alkyl groups.

R^{c} and R^{d} can be independently selected from H, substituted or unsubstituted C₁ to C₂ alkyl groups, methyl, ethoxy, hydroxyethyl, and hydroxymethyl.

The acyclic polyamides of the present invention may comprise a majority of the repeating unit of Formula XX IX or Formula XXX, or the acyclic polyamides can comprise at least about 50 mole % of the repeating unit of Formula XXIX or Formula XXX, including at least about 70 mole %, and at least 80 mole %.

Specific examples of repeating units of Formula XXIX or Formula XXX include repeating units derived from N-vinyl-N-methylacetamide, N-vinylacetamide, N-vinyl-N-methylpropionamide, N-vinyl-N-methyl-2-methylpropionamide, N-vinyl-2-methyl-propionamide, N-vinyl-N,N′-dimethylurea, N,N-dimethylacrylamide, methacrylamide and acyclic amides of Formulae XXXI and XXXII:

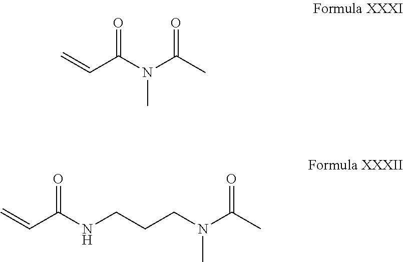

Examples of suitable cyclic amides that can be used to form the cyclic polyamides include α-lactam, β-lactam, γ-lactam, δ-lactam, and ε-lactam. Examples of suitable cyclic polyamides include polymers and copolymers comprising repeating units of Formula XXXIII:

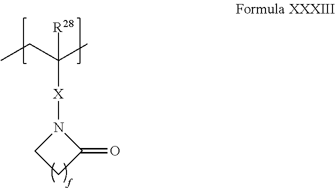

wherein f is a number from 1 to 10, X is a direct bond, —(CO)—, or —(CO)—NH—R^{e}—, wherein R^{e} is a C₁ to C₃ alkyl group and R²⁸ is a hydrogen atom or methyl group. In Formula XXXIII, f may be 8 or less, including 7, 6, 5, 4, 3, 2, or 1. In Formula XXXIII, f may be 6 or less, including 5, 4, 3, 2, or 1, or may be from 2 to 8, including 2, 3, 4, 5, 6, 7, or 8, or may be 2 or 3.

When X is a direct bond, f may be 2. In such instances, the cyclic polyamide may be polyvinylpyrrolidone (PVP).

The cyclic polyamides may comprise 50 mole % or more of the repeating unit of Formula XXXIII, or the cyclic polyamides can comprise at least about 50 mole % of the repeating unit of Formula XXXIII, including at least about 70 mole %, and at least about 80 mole %.

Specific examples of repeating units of Formula XXXIII include repeating units derived from N-vinylpyrrolidone, which forms PVP homopolymers and vinylpyrrolidone copolymers or N-vinylpyrrolidone substituted with hydrophilic substituents such as phosphoryl choline.

The polyamides may also be copolymers comprising cyclic amide, acyclic amide repeating units or copolymers comprising both cyclic and acyclic amide repeating units. Additional repeating units may be formed from monomers selected from hydroxyalkyl(meth)acrylates, alkyl(meth)acrylates or other hydrophilic monomers and siloxane substituted acrylates or methacrylates. Any of the monomers listed as suitable hydrophilic monomers may be used as comonomers to form the additional repeating units. Specific examples of additional monomers which may be used to form polyamides include 2-hydroxyethylmethacrylate, vinyl acetate, acrylonitrile, hydroxypropyl methacrylate, 2-hydroxyethyl acrylate, methyl methacrylate and hydroxybutyl methacrylate, GMMA, PEGS, and the like and mixtures thereof. Ionic monomers may also be included. Examples of ionic monomers include acrylic acid, methacrylic acid, 2-methacryloyloxyethyl phosphorylcholine, 3-(dimethyl(4-vinylbenzyl)ammonio)propane-1-sulfonate (DMVBAPS), 3-((3-acrylamidopropyl)dimethylammonio)propane-1-sulfonate (AMPDAPS), 3-((3-methacrylamidopropyl)dimethylammonio)propane-1-sulfonate (MAMPDAPS), 3-((3-(acryloyloxy)propyl)dimethylammonio)propane-1-sulfonate (APDAPS), methacryloyloxy)propyl)dimethylammonio)propane-1-sulfonate (MAPDAPS).

The reactive composition may comprise both an acyclic polyamide and a cyclic polyamide or copolymers thereof. The acyclic polyamide can be any of those acyclic polyamides described herein or copolymers thereof, and the cyclic polyamide can be any of those cyclic polyamides described herein or copolymers thereof. The polyamide may be selected from the group polyvinylpyrrolidone (PVP), polyvinylmethyacetamide (PVMA), polydimethylacrylamide (PDMA), polyvinylacetamide (PNVA), poly(hydroxyethyl(meth)acrylamide), polyacrylamide, and copolymers and mixtures thereof.

The wetting agents may be made from DMA, NVP, HEMA, VMA, NVA, and combinations thereof. The wetting agents may also be reactive components, as defined herein, by having polymerizable groups, for example, made by the acylation reaction between pendant hydroxyl groups on HEMA repeating units of an internal wetting agent and methacryloyl chloride or methacryloyl anhydride. Other methods of functionalization will be apparent to one skilled in the art.

Such internal wetting agents are disclosed in U.S. Pat. Nos. 6,367,929, 6,822,016, 7,052,131, 7,666,921, 7,691,916, 7,786,185, 8,022,158, and 8,450,387.

Generally, the reactive components within a reactive composition may be dispersed or dissolved in a diluent. Suitable diluents are known in the art or can be easily determined by a person of ordinary skill in the art. For example, when silicone hydrogels are being prepared, suitable diluents are disclosed in WO 03/022321 and U.S. Pat. No. 6,020,445 the disclosures of which are incorporated herein by reference.

Classes of suitable diluents for silicone hydrogel reaction mixtures include alcohols having 2 to 20 carbons, amides having 10 to 20 carbon atoms derived from primary amines and carboxylic acids having 8 to 20 carbon atoms. Primary and tertiary alcohols are preferred. Preferred classes include alcohols having 5 to 20 carbons and carboxylic acids having 10 to 20 carbon atoms.

Specific diluents which may be used include 1-ethoxy-2-propanol, diisopropylaminoethanol, isopropanol, 3,7-dimethyl-3-octanol, 1-decanol, 1-dodecanol, 1-octanol, 1-pentanol, 2-pentanol, 1-hexanol, 2-hexanol, 2-octanol, 3-methyl-3-pentanol, tert-amyl alcohol, tert-butanol, 2-butanol, 1-butanol, 2-methyl-2-pentanol, 2-propanol, 1-propanol, ethanol, 2-ethyl-1-butanol, (3-acetoxy-2-hydroxypropyloxy)propylbis(trimethylsiloxy) methylsilane, 1-tert-butoxy-2-propanol, 3,3-dimethyl-2-butanol, tert-butoxyethanol, 2-octyl-1-dodecanol, decanoic acid, octanoic acid, dodecanoic acid, 2-(diisopropylamino)ethanol mixtures thereof and the like.

Preferred diluents include 3,7-dimethyl-3-octanol, 1-dodecanol, 1-decanol, 1-octanol, 1-pentanol, 1-hexanol, 2-hexanol, 2-octanol, 3-methyl-3-pentanol, 2-pentanol, t-amyl alcohol, tert-butanol, 2-butanol, 1-butanol, 2-methyl-2-pentanol, 2-ethyl-1-butanol, ethanol, 3,3-dimethyl-2-butanol, 2-octyl-1-dodecanol, decanoic acid, octanoic acid, dodecanoic acid, mixtures thereof and the like.

More preferred diluents include 3,7-dimethyl-3-octanol, 1-dodecanol, 1-decanol, 1-octanol, 1-pentanol, 1-hexanol, 2-hexanol, 2-octanol, 1-dodecanol, 3-methyl-3-pentanol, 1-pentanol, 2-pentanol, t-amyl alcohol, tert-butanol, 2-butanol, 1-butanol, 2-methyl-2-pentanol, 2-ethyl-1-butanol, 3,3-dimethyl-2-butanol, 2-octyl-1-dodecanol, mixtures thereof and the like.

Suitable diluents for non-silicone containing reaction compositions include glycerin, ethylene glycol, ethanol, methanol, ethyl acetate, methylene chloride, polyethylene glycol, polypropylene glycol, low number average molecular weight polyvinylpyrrolidone (PVP), such as disclosed in U.S. Pat. Nos. 4,018,853, 4,680,336 and 5,039,459, including, but not limited to boric acid esters of dihydric alcohols, combinations thereof and the like.

Mixtures of diluents may be used. The diluents may be used in amounts up to about 55% by weight of the total of all components in the reactive composition. More preferably the diluent is used in amounts less than about 45% and more preferably in amounts between about 15 and about 40% by weight of the total of all components in the reactive composition.

In a preferred aspect, the crosslinked substrate network of the invention may be a silicone hydrogel (containing covalently bound activatable free radical initiators such as MAPO groups) and the grafting composition may provide, following polymerization, a hydrophilic grafted material (which may optionally be charged), for instance comprising poly(N,N-dimethylacrylamide) (PDMA), polymerized polyethylene glycol mono-methacrylate (e.g., having number average molecular weight from about 300 to about 1000) (poly(mPEG)), a copolymer of 2-hydroxyethyl methacrylate and methacrylic acid, 2-(methacryloyloxy)ethyl (2-(trimethylammonio)ethyl) phosphate (MPC). Such grafted polymer networks may exhibit improved biocompatibility and biometrics when used in ophthalmic devices.

The crosslinked substrate network may be a silicone hydrogel (containing covalently bound activatable free radical initiators such as MAPO groups) and the grafting composition provides, following polymerization, a hydrophobic siloxane containing material. Such grafted polymeric networks may display modified physical, mechanical, and surface properties, such as oxygen gas permeability (Dk), modulus, and coefficient of friction, respectively, as well as improved handling such as insertion and removal of a contact lens in an eye.

The crosslinked substrate network may be a conventional hydrogel (e.g., comprising a copolymer of 2-hydroxyethyl methacrylate and methacrylic acid and containing MAPO groups) and the grafting composition provides, following polymerization, a hydrophilic grafted material (which may optionally be charged), such as a polyamide. Examples include PDMA, polyvinylpyrrolidone (PVP), poly(N-vinyl N-methyl acetamide) (PVMA), and copolymers thereof. Such grafted polymer networks may exhibit improved biocompatibility and biometrics, for instance when used in ophthalmic devices.

The crosslinked substrate network may be a conventional hydrogel (e.g., a copolymer of 2-hydroxyethyl methacrylate and methacrylic acid and containing MAPO groups) and the grafting composition provides, following polymerization, a hydrophobic siloxane containing material. Such grafted polymeric networks may exhibit desirable physical and mechanical properties, such as oxygen gas permeability (Dk) and modulus, as well as improved biocompatibility and handling.

For ophthalmic devices, such as contact lenses, that contain one or more silicone containing component, the silicone-containing component(s) may preferably be present in amounts up to about 95 weight %, or from about 10 to about 80, or from about 20 to about 70 weight %, based upon all reactive components present, including in the reactive composition and the reactive second composition. Suitable hydrophilic components may preferably be present in amounts from about 10 to about 60 weight %, or from about 15 to about 50 weight %, or from about 20 to about 40 weight %, based upon all reactive components present, including in the reactive composition and the grafting composition.

It should be noted that additional, optional, steps may be included in the process for making the polymer compositions of the invention. For instance, following step (b), an ink or dye may be added to the crosslinked substrate network. Then, the remaining steps (step (c) etc.) may be carried out. This allows for an ink or dye to be sandwiched within the grafted polymeric network.

For ophthalmic devices, such as contact lenses, the crosslinked substrate network is preferably a silicone hydrogel with a balance of properties that makes them desirable. These properties include water content, haze, contact angle, modulus, oxygen permeability, lipid uptake, lysozyme uptake and PQ1 uptake. Examples of preferred properties are as follows. All values are prefaced by “about,” and the ophthalmic devices may have any combination of the listed properties:
Water content: at least 20%, or at least 25%
Haze: 30% or less, or 10% or less
Dynamic contact angle (DCA (°)): 1000 or less, or 50° or less
Modulus (psi): 120 or less, or 80 to 120
Oxygen permeability (Dk (barrers)): at least 80, or at least 100, or at least 150, or at least 200
Elongation to Break: at least 100
   For ionic silicon hydrogels, the following properties may also be preferred (in addition to those recited above):
Lysozyme uptake (μg/lens): at least 100, or at least 150, or at least 500, or at least 700
Polyquaternium-1 (PQ1) uptake (%): 15 or less, or 10 or less, or 5 or less

Finished ophthalmic devices may be manufactured by various techniques. For instance, in the case of hydrogel contact lenses, the reactive composition described above may be cured in a mold, or formed via spincasting or static casting. Spincasting methods are disclosed in U.S. Pat. Nos. 3,408,429 and 3,660,545, and static casting methods are disclosed in U.S. Pat. Nos. 4,113,224 and 4,197,266. In one embodiment, the contact lenses of this invention are formed by the direct molding of the hydrogels, which is economical, and enables precise control over the final shape of the hydrated contact lens. For this method, the reactive composition is placed in a mold having the desired shape and the reactive composition is subjected to conditions as described above whereby the reactive components polymerize to produce the crosslinked substrate network in the approximate shape of the final desired product.

The crosslinked substrate network formed after such curing may be subjected to extraction to remove unreacted components and release the crosslinked substrate network from the contact lens mold. The crosslinked substrate network may then be immersed in the grafting composition (which may optionally contain a diluent), and sufficient time is allowed to permit at the reactive composition to diffuse into the crosslinked substrate network to the desired level. Thereafter, the suspension is irradiated as described above to form the grafted product, and the contact lenses may then be extracted to remove unreacted components.

Extractions of the crosslinked substrate network and the contact lens may be done using conventional extraction fluids, such organic solvents, such as alcohols or may be extracted using aqueous solutions. Aqueous solutions are solutions which comprise water. The aqueous solutions may comprise at least about 30 weight % water, or at least about 50 weight % water, or at least about 70% water or at least about 90 weight % water.

Extraction may be accomplished, for example, via immersion of the crosslinked substrate network or the contact lens in an aqueous solution or exposing the material to a flow of an aqueous solution. Extraction may also include, for example, one or more of: heating the aqueous solution; stirring the aqueous solution; increasing the level of release aid in the aqueous solution to a level sufficient to cause release of the crosslinked substrate network from the mold; mechanical or ultrasonic agitation; and incorporating at least one leach aid in the aqueous solution to a level sufficient to facilitate adequate removal of unreacted components from the crosslinked substrate network or the contact lens. The foregoing may be conducted in batch or continuous processes, with or without the addition of heat, agitation or both.

Some embodiments may also include the application of physical agitation to facilitate leach and release. For example, the crosslinked substrate network mold part to which the crosslinked substrate network is adhered may be vibrated or caused to move back and forth within an aqueous solution. Other embodiments may include ultrasonic waves through the aqueous solution.

Contact lenses may be sterilized by known means such as, but not limited to, autoclaving.

### CLAUSES

For reasons of completeness, various aspects of the disclosure are set forth in the following numbered clauses.

Clause 1. An ophthalmic device formed by a process comprising:
a. providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
b. subjecting the reactive composition to a first activation step such that the reactive composition polymerizes therein to form a crosslinked substrate network containing a covalently bound activatable free radical initiator;
c. deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions;
d. contacting the crosslinked substrate network with a grafting composition containing one or more ethylenically unsaturated compounds, wherein the contacting is conducted under conditions such that the grafting composition penetrates into the crosslinked substrate network; and
e. activating the retained covalently bound activatable free radical initiator such that the grafting composition polymerizes with the crosslinked substrate network, thereby forming grafted polymeric networks, outside of the selective regions and optionally partially within the selective regions.

Clause 2. The ophthalmic device of clause 1 wherein the deactivating of step (c) varies spatially within the ophthalmic device.

Clause 3. The ophthalmic device of any one of clauses 1 to 2 wherein the deactivating step (c) is achieved by irradiation in an oxygen gas atmosphere.

Clause 4. The ophthalmic device of any one of clauses 1 to 3 wherein the deactivating step (c) is preformed using ultraviolet light having a wavelength between 300 nanometers and 400 nanometers.

Clause 5. The ophthalmic device of clause 4 wherein the grafting step (e) is performed using visible light having a wavelength between 400 nanometers and 450 nanometers.

Clause 6. The ophthalmic device of any one of clauses 1 to 3 wherein the deactivating step (c) is preformed using visible light having a wavelength between 400 nanometers and 500 nanometers.

Clause 7. The ophthalmic device of clause 6 wherein the grafting step (e) is performed using ultraviolet light having a wavelength between 300 nanometers and 400 nanometers.

Clause 8. The ophthalmic device of clause 4 or clause 7 wherein the ultraviolet light has a wavelength between 350 nanometer and 400 nanometers.

Clause 9. The ophthalmic device of clause 5 or clause 6 wherein the visible light has a wavelength between 400 nanometers and 450 nanometers.

Clause 10. The ophthalmic device of clause 1 wherein the grafting composition of step (d) contains a crosslinker.

Clause 11. The ophthalmic device of clause 1 wherein the grafting composition of step (d) is free of a crosslinker.

Clause 12. The ophthalmic device of any one of clauses 1 to 11 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 13. The ophthalmic device of any one of clauses 1 to 12 wherein the one or more ethylenically unsaturated compounds of step (d) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 14. The ophthalmic device of any one of clauses 1 to 13 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

Clause 15. The ophthalmic device of any one of clauses 1 to 14 wherein the polymerization initiator is a bisacylphosphine oxide or a bis(acyl)phosphane oxide.

Clause 16. The ophthalmic device of any one of clauses 1 to 15 that is in the form of a hydrogel and wherein the reactive composition contains one or more silicone-containing components, and the grafting composition contains one or more hydrophilic reactive components.

Clause 17. The ophthalmic device of any one of clauses 1 to 15 that is in the form of a hydrogel and wherein the reactive composition contains one or more hydrophilic reactive components, and the grafting composition contains one or more silicone-containing components.

Clause 18. The ophthalmic device of any one of clauses 1 to 15 wherein the reactive composition, the grafting composition, or both the reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

Clause 19. The ophthalmic device of clause 18 wherein the UV absorber is 2-(2′-hydroxy-5-methacrylyloxyethylphenyl)-2H-benzotriazole.

Clause 20. The ophthalmic device of any one of clauses 1 to 19 wherein the process further comprises: following step (c), extracting the crosslinked substrate network with a solvent and optionally hydrating the extracted crosslinked substrate network with an aqueous solution.

Clause 21. The ophthalmic device of any one of clauses 1 to 20 wherein the process further comprises: following step (e), contacting the crosslinked substrate network with a second grafting composition containing one or more ethylenically unsaturated compounds and activating retained covalently bound activatable free radical initiator such that the second grafting composition polymerizes with the crosslinked substrate network outside of the selective regions and optionally partially within the selective regions.

Clause 22. The ophthalmic device of any one of clauses 1 to 21 wherein the process steps (a) and (b) are performed in a mold assembly comprised of a front mold and a back mold, the front mold and a back mold defining and enclosing a cavity in the shape of the ophthalmic device therebetween, and process steps (c), (d), and (e) are performed in the mold assembly after the back mold has been removed.

Clause 23. The ophthalmic device of any one of clauses 1 to 22 wherein the source of actinic irradiation in steps (c) and (e) includes a plurality of selectively controllable beams of actinic radiation controlled by a digital micro-mirror device according to a predetermined script.

Clause 24. The ophthalmic device of clause 23 wherein the plurality of selectively controllable beams of actinic radiation controlled by the digital micro-mirror devices according to predetermined scripts are directed to one or more surfaces of the ophthalmic device.

Clause 25. The ophthalmic device of any one of clauses 23 to 24 wherein the digital micro-mirror device includes an illumination source containing at least one light emitting diode.

Clause 26. The ophthalmic device of any one of clauses 1 to 25 wherein the ophthalmic device is selected from the group consisting of a contact lens, an intraocular lens, a punctal plug, and an ocular insert.

Clause 27. The ophthalmic device of clause 26 wherein the ophthalmic device is a contact lens or an intraocular lens.

Clause 28. An ophthalmic device comprised of a reaction product of a composition comprising:
a. a crosslinked substrate network, wherein at least a portion of the covalently bound activatable free radical initiators are deactivated in one or more selective regions of the crosslinked substrate network such that the crosslinked substrate network contains retained covalently bound activatable free radical initiators outside of the one or more selective regions and optionally within the one or more selective regions; and
b. a grafting composition containing one or more ethylenically unsaturated compounds, wherein the grafting composition is localized in the crosslinked substrate network where there are retained covalently bound activatable free radical initiators.

Clause 29. The ophthalmic device of clause 28 wherein the concentration of the retained covalently bound activatable free radical initiators varies spatially within crosslinked substrate network.

Clause 30. The ophthalmic device of any one of clauses 28 to 29 wherein the crosslinked substrate network is the reaction product of a reactive composition comprising: (i) a polymerization initiator that is capable, upon a first activation, of forming two or more free radical groups, at least one of which is further activatable by subsequent activation; (ii) one or more ethylenically unsaturated compounds; and (iii) a crosslinker.

Clause 31. The ophthalmic device of clause 30 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

Clause 32. The ophthalmic device of clause 31 wherein the polymerization initiator is a bisacylphosphine oxide or a bis(acyl)phosphane oxide.

Clause 33. The ophthalmic device of clause 28 wherein the covalently bound activatable free radical initiators are selected from the group consisting of monoacylphosphine oxides, bisacylphosphine oxides, and combinations thereof.

Clause 34. The ophthalmic device of clause 28 wherein the crosslinked substrate network is formed by the thermal free radical polymerization of a reactive monomer mixture comprising at least one reactive component having at least one pendant group selected from the group consisting of monoacylphosphine oxide, bisacylphosphine oxide, and combinations thereof.

Clause 35. The ophthalmic device of clause 34 further comprising a monoacylphosphine oxide compound having a refractive index moiety, a bisacylphosphine oxide compound having a refractive index moiety, or combinations thereof.

Clause 36. The ophthalmic device of clause 35 wherein the refractive index moiety is a polyamide.

Clause 37. The ophthalmic device of clause 36 wherein the polyamide comprises polyvinylpyrrolidone (PVP), polyvinylmethyacetamide (PVMA), polydimethylacrylamide (PDMA), polyvinylacetamide (PNVA), poly(hydroxyethyl(meth)acrylamide), polyacrylamide, a copolymer of two or more thereof, or a combination of two or more thereof.

Clause 38. The ophthalmic device of clause 35 wherein the light absorbing moiety comprises a static dye, a photochromic dye, a thermochromic dye, a leuco dye, or a combination of two or more thereof.

Clause 39. The ophthalmic device of any one of clauses 35 to 37 wherein the crosslinked substrate network is formed by a combination of thermal polymerization and photopolymerization having the following steps:
a. thermally polymerizing the reactive composition, comprising at least one ethylenically unsaturated compound, at least one reactive component selected from the group consisting of a monoacylphosphine oxide monomer (MAPO-M), a bisacylphosphine oxide monomer (MAPO-M), or combinations thereof; and a thermal crosslinker, to form a precursor crosslinked substrate network, wherein the monoacylphosphine oxide compounds having refractive index or light absorbing moieties and bisacylphosphine oxide compounds having refractive index or light absorbing moieties are spatially dispersed within a precursor crosslinked substrate network and wherein the reactive composition has not been fully polymerized;
b. irradiating the precursor crosslinked substrate network in preselected regions, thereby initiating the free radical polymerization from the dispersed monoacylphosphine oxide compounds having refractive index or light absorbing moieties and bisacylphosphine oxide compounds having refractive index or light absorbing moieties, thereby incorporating the refractive index or light absorbing moieties into the precursor crosslinked substrate network;
c. thermally polymerizing until the reactive composition has been completely polymerized; and optionally
d. extracting the unreacted monoacylphosphine oxide compounds having refractive index or light absorbing moieties and bisacylphosphine oxide compounds having refractive index or light absorbing moieties with a solvent;
wherein steps (a) through (d) are conducted under conditions that preserve the reactivity of the repeating units derived from the reactive components having at least one pendant group selected from the group consisting of monoacylphosphine oxide, bisacylphosphine oxide, and combinations thereof in the precursor crosslinked substrate network and the crosslinked substrate network.

Clause 40. The ophthalmic device of clause 39 wherein the process step (a) is performed at a temperature between 60° C. and 100° C.

Clause 41. The ophthalmic device of any one of clauses 39 to 40 wherein the process step (a) is performed using azobisisobutyronitrile as the thermal initiator.

Clause 42. The ophthalmic device of any one of clauses 39 to 41 wherein the source of actinic irradiation in step (b) includes a plurality of selectively controllable beams of actinic radiation controlled by a digital micro-mirror device according to a predetermined script.

Clause 43. The ophthalmic device of clause 42 wherein the digital micro-mirror device includes an illumination source containing at least one light emitting diode.

Clause 44. The ophthalmic device of clause 43 wherein the light emitting diode emits radiation having one or more wavelengths in the range of 365 nanometers to 450 nanometers.

Clause 45. The ophthalmic device of any one of clauses 28 to 44 wherein the one or more ethylenically unsaturated compounds in the grafting composition and the reactive composition comprise polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 46. The ophthalmic device of any one of clauses 28 to 44 wherein the crosslinked substrate network is formed from one or more silicone-containing components and the grafting composition contains hydrophilic reactive components.

Clause 47. The ophthalmic device of any one of clauses 28 to 44 wherein the crosslinked substrate network is formed from one or more hydrophilic reactive components and the grafting composition contains one or more silicone-containing components.

Clause 48. The ophthalmic device of any one of clauses 28 to 44 wherein the crosslinked substrate network is formed from one or more hydrophilic reactive components and the grafting composition contains hydrophilic reactive components.

Clause 49. The ophthalmic device of any one of clauses 28 to 44 wherein the crosslinked substrate network is formed from one or more silicone-containing components and the grafting composition contains one or more silicone-containing components.

Clause 50. The ophthalmic device of any one of clauses 28 to 49 wherein the crosslinked substrate network, the grafting composition, or both the crosslinked substrate network and the grafting composition contain one or more additives selected from UV absorbers, photochromic compounds, pharmaceutical compounds, nutraceutical compounds, antimicrobial compounds, tints, pigments, dyes, dyes, release agents, and wetting agents.

Clause 51. The ophthalmic device of clause 50 wherein the UV absorber is 2-(2′-hydroxy-5-methacrylyloxyethylphenyl)-2H-benzotriazole.

Clause 52. The ophthalmic device of any one of clauses 28 to 51 wherein the ophthalmic device is selected from the group consisting of a contact lens, an intraocular lens, a punctal plug, and an ocular insert.

Clause 53. The ophthalmic device of clause 52 wherein the ophthalmic device is a contact lens or an intraocular lens.

Clause 54. A process for making an ophthalmic device, the process comprising:
a. providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
b. subjecting the reactive composition to a first activation step such that the reactive composition polymerizes therein to form a crosslinked substrate network containing a covalently bound activatable free radical initiator;
c. deactivating at least a portion of the covalently bound activatable free radical initiator at one or more selective regions of the crosslinked substrate network such that the crosslinked substrate network contains retained covalently bound activatable free radical initiator outside of the one or more selective regions and optionally within the one or more selective regions;
d. contacting the crosslinked substrate network with a grafting composition containing one or more ethylenically unsaturated compounds, wherein the contacting is conducted under conditions such that the grafting composition penetrates into the crosslinked substrate network; and
e. activating the retained covalently bound activatable free radical initiator such that the grafting composition polymerizes with the crosslinked substrate network, thereby forming grafted polymeric networks, outside of the selective regions and optionally partially within the selective regions.

Clause 55. The ophthalmic device of clause 54 wherein the deactivating of step (c) varies spatially within the ophthalmic device.

Clause 56. The ophthalmic device of any one of clauses 54 to 55 wherein the deactivating step (c) is achieved by irradiation in an oxygen gas atmosphere.

Clause 57. The ophthalmic device of any one of clauses 54 to 56 wherein the deactivating step (c) is preformed using ultraviolet light having a wavelength between 300 nanometers and 400 nanometers.

Clause 58. The ophthalmic device of clause 57 wherein the grafting step (e) is performed using visible light having a wavelength between 400 nanometers and 450 nanometers.

Clause 59. The ophthalmic device of any one of clauses 54 to 56 wherein the deactivating step (c) is preformed using visible light having a wavelength between 400 nanometers and 500 nanometers.

Clause 60. The ophthalmic device of clause 59 wherein the grafting step (e) is performed using ultraviolet light having a wavelength between 300 nanometers and 400 nanometers.

Clause 61. The ophthalmic device of clause 57 or clause 60 wherein the ultraviolet light has a wavelength between 350 nanometer and 400 nanometers.

Clause 62. The ophthalmic device of clause 58 or clause 59 wherein the visible light has a wavelength between 400 nanometers and 450 nanometers.

Clause 63. The process of clause 54 wherein the grafting composition of step (e) contains a crosslinker.

Clause 64. The process of clause 54 wherein the grafting composition of step (e) is free of a crosslinker.

Clause 65. The process of any one of clauses 54 to 64 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 66. The process of any one of clauses 54 to 65 wherein the one or more ethylenically unsaturated compounds of step (e) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 67. The process of any one of clauses 54 to 66 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

Clause 68. The process of clause 67 wherein the polymerization initiator is a bisacylphosphine oxide or a bis(acyl)phosphane oxide.

Clause 69. The process of any one of clauses 54 to 67 that is in the form of a hydrogel and wherein the reactive composition contains one or more silicone-containing components and the grafting composition contains one or more hydrophilic reactive components.

Clause 70. The process of any one of clauses 54 to 67 that is in the form of a hydrogel and wherein the reactive composition contains one or more hydrophilic reactive components and the grafting composition contains one or more silicone-containing components.

Clause 71. The process of any one of clauses 54 to 70 wherein the reactive composition, the grafting composition, or both the reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

Clause 72. The process of clause 71 wherein the UV absorber is 2-(2′-hydroxy-5-methacrylyloxyethylphenyl)-2H-benzotriazole.

Clause 73. The process of any one of clauses 54 to 72 wherein the process further comprises: following step (e), contacting the crosslinked substrate network with a second grafting composition containing one or more ethylenically unsaturated compounds and activating retained covalently bound activatable free radical initiator such that the second grafting composition polymerizes with the crosslinked substrate network outside of the selective regions and optionally partially within the selective regions.

Clause 74. The process of any one of clauses 54 to 73 wherein the process steps (a) and (b) are performed in a mold assembly comprised of a front mold and a back mold, the front mold and a back mold defining and enclosing a cavity in the shape of the ophthalmic device therebetween, and process steps (c), (d), and (e) are performed in the mold assembly after the back mold has been removed.

Clause 75. A process for making an ophthalmic device, the process comprising:
a. providing a reactive composition containing: (i) a thermal initiator; (ii) one or more ethylenically unsaturated compounds; (iii) a crosslinker; (iv) at least one reactive component having at least one pendant group selected from the group consisting of monoacylphosphine oxide, bisacylphosphine oxide, and combinations thereof; (v) at least one reactive component selected from the group consisting of monoacylphosphine oxide compounds having refractive index or light absorbing moieties, bisacylphosphine oxide compounds having refractive index or light absorbing moieties, and combinations thereof;
b. subjecting the reactive composition to a thermal polymerization to form a precursor crosslinked substrate network, wherein the monoacylphosphine oxide compounds having refractive index or light absorbing moieties and bisacylphosphine oxide compounds having refractive index or light absorbing moieties are spatially dispersed within the precursor crosslinked substrate network and wherein the reactive composition has not been fully polymerized;
c. irradiating the precursor crosslinked substrate network in preselected regions, thereby initiating free radical polymerizations from the dispersed monoacylphosphine oxide compounds having refractive index or light absorbing moieties and bisacylphosphine oxide compounds having refractive index or light absorbing moieties, thereby incorporating the refractive index or light absorbing moieties into the precursor crosslinked substrate network in the preselected regions;
d. subjecting the reactive composition to the thermal polymerization until the reactive composition has been completely polymerized to form a crosslinked substrate network having covalently bound monoacylphosphine oxide or bisacylphosphine oxide groups; and optionally
e. extracting the unreacted monoacylphosphine oxide compounds having refractive index or light absorbing moieties and bisacylphosphine oxide compounds having refractive index or light absorbing moieties with a solvent;
f. deactivating at least a portion of the covalently bound monoacylphosphine oxide or bisacylphosphine oxide groups at one or more selective regions of the crosslinked substrate network such that the crosslinked substrate network contains retained covalently bound monoacylphosphine oxide or bisacylphosphine oxide groups outside of the one or more selective regions and optionally within the one or more selective regions;
g. contacting the crosslinked substrate network with a grafting composition containing one or more ethylenically unsaturated compounds, wherein the contacting is conducted under conditions such that the grafting composition penetrates into the crosslinked substrate network; and
h. activating the retained covalently bound monoacylphosphine oxide or bisacylphosphine oxide groups such that the grafting composition polymerizes with the crosslinked substrate network, thereby forming grafted polymeric networks, outside of the selective regions and optionally partially within the selective regions.
wherein steps (a) through (h) are conducted under conditions that preserve the reactivity of the covalently bound monoacylphosphine oxide or bisacylphosphine oxide groups in the precursor crosslinked substrate network and the crosslinked substrate network until said covalently bound monoacylphosphine oxide or bisacylphosphine oxide groups are intentionally irradiated or activated.

Clause 76. The ophthalmic device of clause 75 wherein the process step (b) is performed at a temperature between 60° C. and 100° C.

Clause 77. The ophthalmic device of any one of clauses 75 to 76 the process step (b) is performed using azobisisobutyronitrile as the thermal initiator.

Clause 78. The process of any one of clauses 75 to 77 wherein the source of actinic irradiation in step (c) includes a plurality of selectively controllable beams of actinic radiation controlled by a digital micro-mirror device according to a predetermined script.

Clause 79. The process of clause 78 wherein the digital micro-mirror device includes an illumination source containing at least one light emitting diode.

Clause 80. The process of clause 79 wherein the light emitting diode emits radiation having one or more wavelengths in the range of 365 nanometers to 450 nanometers.

Clause 81. The process of any one of clauses 75 to 80 wherein the deactivating of step (f) varies spatially within the ophthalmic device.

Clause 82. The process of any one of clauses 75 to 81 wherein the deactivating step (c) is achieved by irradiation in an oxygen gas atmosphere.

Clause 83. The process of any one of clauses 75 to 82 wherein the deactivating step (c) is preformed using ultraviolet light having a wavelength between 300 nanometers and 400 nanometers.

Clause 84. The process of clause 83 wherein the grafting step (e) is performed using visible light having a wavelength between 400 nanometers and 450 nanometers.

Clause 85. The process of any one of clauses 75 to 82 wherein the deactivating step (c) is preformed using visible light having a wavelength between 400 nanometers and 500 nanometers.

Clause 86. The process of clause 85 wherein the grafting step (e) is performed using ultraviolet light having a wavelength between 300 nanometers and 400 nanometers.

Clause 87. The process of clause 83 or clause 86 wherein the ultraviolet light has a wavelength between 350 nanometer and 400 nanometers.

Clause 88. The process of clause 84 or clause 85 wherein the visible light has a wavelength between 400 nanometers and 450 nanometers.

Clause 89. The process of any of clauses 75 to 88 wherein the grafting composition of step (g) contains a crosslinker.

Clause 90. The process of any of clauses 75 to 88 wherein the grafting composition of step (g) is free of a crosslinker.

Clause 91. The process of any one of clauses 75 to 90 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 92. The process of any one of clauses 75 to 91 the one or more ethylenically unsaturated compounds of step (g) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 93. The process of any one of clauses 75 to 92 that is in the form of a hydrogel and wherein the reactive composition contains one or more silicone-containing components, and the grafting composition contains one or more hydrophilic reactive components.

Clause 94. The process of any one of clauses 75 to 92 that is in the form of a hydrogel and wherein the reactive composition contains one or more hydrophilic reactive components, and the grafting composition contains one or more silicone-containing components.

Clause 95. The process of any one of clauses 75 to 94 wherein the reactive composition, the grafting composition, or both the reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

Clause 96. The process of clause 95 wherein the UV absorber is 2-(2′-hydroxy-5-methacrylyloxyethylphenyl)-2H-benzotriazole.

Clause 97. The process of any one of clauses 75 to 96 wherein the process further comprises: following step (h), contacting the crosslinked substrate network with a second grafting composition containing one or more ethylenically unsaturated compounds and activating retained covalently bound activatable free radical initiator such that the second grafting composition polymerizes with the crosslinked substrate network outside of the selective regions and optionally partially within the selective regions.

Clause 98. The process of any one of clauses 75 to 97 wherein the process steps (a) through (f) excluding optional step (e) are performed in a mold assembly comprised of a front mold and a back mold, the front mold and the back mold defining and enclosing a cavity in the shape of the ophthalmic device therebetween, and process steps (g) and (h) are performed in the mold assembly after the back mold has been removed.

Clause 99. The ophthalmic device of clause 1 or clause 28 wherein the selective regions form a pattern.

Clause 100. The ophthalmic device of clause 1 or clause 28 wherein the selective regions form a fiduciary marker.

Clause 101. The ophthalmic device of clause 1 or clause 28 wherein the selective regions form a barcode.

Clause 102. An ophthalmic device formed by a process comprising:
(a) providing a first reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
(b) subjecting the first reactive composition to a first activation step such that the first reactive composition polymerizes therein to form a crosslinked substrate network containing a covalently bound activatable free radical initiator;
(c) contacting the crosslinked substrate network with a grafting composition containing one or more ethylenically unsaturated compounds, wherein the contacting is conducted under conditions such that the grafting composition penetrates into the crosslinked substrate network; and
(d) activating the covalently bound activatable free radical initiator at one or more selective regions of the crosslinked substrate network such that the grafting composition polymerizes with the crosslinked substrate network at the selective regions,
   wherein steps (a) and (b) are performed in a mold assembly comprised of a front mold and a back mold, the front mold and the back mold defining and enclosing a cavity in the shape of the ophthalmic device therebetween, and steps (c) and (d) are performed in the mold assembly after the back mold has been removed.

Clause 103. A process for making an ophthalmic device, the process comprising:
(a) providing a first reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
(b) subjecting the first reactive composition to a first activation step such that the first reactive composition polymerizes therein to form a crosslinked substrate network containing a covalently bound activatable free radical initiator;
(c) contacting the crosslinked substrate network with a grafting composition containing one or more ethylenically unsaturated compounds, wherein the contacting is conducted under conditions such that the grafting composition penetrates into the crosslinked substrate network; and
(d) activating the covalently bound activatable free radical initiator of the crosslinked substrate network such that the grafting composition polymerizes therein with the crosslinked substrate network,
   wherein steps (a) and (b) are performed in a mold assembly comprised of a front mold and a back mold, the front mold and the back mold defining and enclosing a cavity in the shape of the ophthalmic device therebetween, and steps (c) and (d) are performed in the mold assembly after the back mold has been removed.

Clause 104. The ophthalmic device of clause 102 or the process of clause 103 wherein the grafting composition of step (c) contains a crosslinker.

Clause 105. The ophthalmic device of clause 102 or the process of clause 103 wherein the grafting composition of step (c) is free of a crosslinker.

Clause 106. The ophthalmic device of clause 102 or the process of clause 103 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 107. The ophthalmic device of clause 102 or clause 103 wherein the one or more ethylenically unsaturated compounds of step (c) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

Clause 108. The ophthalmic device of clause 102 or clause 103 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

Clause 109. The ophthalmic device of clause 102 or clause 103 wherein the polymerization initiator is a bisacylphosphine oxide or a bis(acyl)phosphane oxide.

Clause 110. The ophthalmic device of clause 102 or clause 103 that is in the form of a hydrogel and wherein the first reactive composition contains one or more silicone-containing components and the grafting composition contains one or more hydrophilic reactive components.

Clause 111. The ophthalmic device of clause 102 or clause 103 that is in the form of a hydrogel and wherein the first reactive composition contains one or more hydrophilic reactive components and the grafting composition contains one or more silicone-containing components.

Clause 112. The ophthalmic device of clause 102 or clause 103 wherein the first reactive composition, the grafting composition, or both the first reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

Clause 113. The ophthalmic device of clause 102 or clause 103 selected from the group consisting of a contact lens, an intraocular lens, a punctal plug and an ocular insert.

Clause 114. An ophthalmic device formed by a process comprising:
(a) providing a crosslinked substrate network having pendant groups selected from bisacylphosphine oxide groups, monoacylphosphine oxide groups, and combinations thereof and having light absorbers selected from ultraviolet light absorbers, visible light absorbers, and combinations thereof
(b) deactivating the pendant groups with light having a wavelength that is partially absorbed by the light absorber;
(c) contacting the crosslinked substrate network with a grafting composition containing one or more ethylenically unsaturated compounds;
(d) activating the pendant groups with light having a wavelength that is not substantially absorbed by the light absorber;
   wherein steps (a), and (b) are optionally performed in a mold assembly comprised of a front mold and a back mold, the front mold and the back mold defining and enclosing a cavity in the shape of the ophthalmic device therebetween; steps (c) and (d) are optionally performed in the mold assembly after the back mold has been removed; and step (b) is optionally performed from above and below the mold assembly.

Clause 115. An ophthalmic device formed by a process comprising:
(a) providing a thermally cured crosslinked substrate network having pendant bisacylphosphine oxide groups;
(b) converting the bisacylphosphine oxide groups into monoacylphosphine oxide groups in first selected regions of the thermally cured crosslinked substrate network;
(c) deactivating both bisacylphosphine oxide groups and monoacylphosphine oxide groups in second selected regions of the thermally cured crosslinked substrate network;
(d) contacting the thermally cured crosslinked substrate network with a first grafting composition containing one or more ethylenically unsaturated compounds;
(e) activating the bisacylphosphine oxide groups such that the first grafting composition polymerizes with the thermally cured crosslinked substrate network;
(f) optionally extracting the first grafting composition from the thermally cured crosslinked substrate network;
(g) contacting the thermally cured crosslinked substrate network with a second grafting composition containing one or more ethylenically unsaturated compounds;
(h) activating the monoacylphosphine oxide groups such that the second grafting composition polymerizes with the thermally cured crosslinked substrate network;
   wherein steps (a), (b), and (c) are optionally performed in a mold assembly comprised of a front mold and a back mold, the front mold and the back mold defining and enclosing a cavity in the shape of the ophthalmic device therebetween, and steps (d) through (h) are optionally performed in the mold assembly after the back mold has been removed.

### EXAMPLES

The contact lens diameter (DM) was measured on a calibrated Van Keuren micro optical comparator equipment equipped with Mitutoyo digimatic micrometer heads. The contact lens was placed concave side down into a crystal cell completely filled with borate buffered packing solution. A cap was placed onto the cell ensuring that no air is trapped underneath. The cell was then placed on the comparator stage and the lens image brought into focus and aligned so that one edge of the lens touched the center line on the screen. The first edge was marked, the lens moved along its diameter until the second edge is touching the center line on the screen, and then, the second edge is marked by pushing the data button again. Typically, two diameter measurements are made, and the average reported in the data tables.

Water content (WC) was measured gravimetrically. Lenses were equilibrated in packing solution for 24 hours. Each of three test lens is removed from packing solution using a sponge tipped swab and placed on blotting wipes which have been dampened with packing solution. Both sides of the lens are contacted with the wipe. Using tweezers, the test lens is placed in a tared weighing pan and weighed. The two more sets of samples are prepared and weighed. All weight measurements were done in triplicate, and the average of those values used in the calculations. The wet weight is defined as the combined weight of the pan and wet lenses minus the weight of the weighing pan alone.

The dry weight was measured by placing the sample pans in a vacuum oven which has been preheated to 60° C. for 30 minutes. Vacuum was applied until the pressure reaches at least 1 inch of Hg is attained; lower pressures are allowed. The vacuum valve and pump are turned off and the lenses are dried for at least 12 hours, typically overnight. The purge valve is opened allowing dry air or dry nitrogen gas to enter. The oven is allowed reach atmospheric pressure. The pans are removed and weighed. The dry weight is defined as the combined weight of the pan and dry lenses minus the weight of the weighing pan alone. The water content of the test lens was calculated as follows: % water content=(wet weight−dry weight)/wet weight×100. The average and standard deviation of the water content were calculated, and the average value reported as the percent water content of the test lens.

The grafted lens weight gain was calculated from the average dry weight of the grafted lens minus the average dry weight of the substrate lens and expressed as a percentage. Both the grafted lens and the substrate lens were equilibrated in deionized water for several hours to remove any residual salts. Typically, at least three lenses are weighed and averaged for each sample.

The refractive index (RI) of a contact lens was measured by a Leica ARIAS 500 Abbe refractometer in manual mode or by a Reichert ARIAS 500 Abbe refractometer in automatic mode with a prism gap distance of 100 microns. The instrument was calibrated using deionized water at 20° C. (+/−0.2° C.). The prism assembly was opened and the test lens placed on the lower prism between the magnetic dots closest to the light source. If the prism is dry, a few drops of saline were applied to the bottom prism. The front curve of the lens was against the bottom prism. The prism assembly was then closed. After adjusting the controls so that the shadow line appeared in the reticle field, the refractive index was measured. The RI measurement was made on five test lenses. The average RI calculated from the five measurements was recorded as the refractive index as well as its standard deviation.

Oxygen permeability (Dk) was determined by the polarographic method generally described in ISO 9913-1:1996 and ISO 18369-4:2006, but with the following modifications. The measurement was conducted at an environment containing 2.1% oxygen created by equipping the test chamber with nitrogen and air inputs set at the appropriate ratio, for example, 1800 mL/min of nitrogen and 200 mL/min of air. The t/Dk is calculated using the adjusted oxygen concentration. Borate buffered saline was used. The dark current was measured by using a pure humidified nitrogen environment instead of applying MMA lenses. The lenses were not blotted before measuring. Four lenses were stacked instead of using lenses of various thickness (t) measured in centimeters. A curved sensor was used in place of a flat sensor; radius was 7.8 mm. The calculations for a 7.8 mm radius sensor and 10% (v/v) air flow are as follows:

Dk/t=(measured current−dark current)×(2.97×10−8 mL O₂/(μA-sec-cm²-mm Hg)

The edge correction was related to the Dk of the material.

For all Dk values less than 90 barrers:

$$t / Dk ( edge corrected ) = [ 1 + ( 5.88 \times t ) ] \times ( t / Dk )$$

For Dk values between 90 and 300 barrers:

$$t / Dk ( edge corrected ) = [ 1 + ( 3.56 \times t ) ] \times ( t / Dk )$$

For Dk values greater than 300 barrers:

$$t / Dk ( edge corrected ) = [ 1 + ( 3.16 \times t ) ] \times ( t / Dk )$$

Non-edge corrected Dk was calculated from the reciprocal of the slope obtained from the linear regression analysis of the data wherein the x variable was the center thickness in centimeters and the y variable was the t/Dk value. On the other hand, edge corrected Dk (EC Dk) was calculated from the reciprocal of the slope obtained from the linear regression analysis of the data wherein the x variable was the center thickness in centimeters and the y variable was the edge corrected t/Dk value. The resulting Dk value was reported in barrers.

Wettability of lenses was determined by a modified Wilhelmy plate method using a calibrated Kruss K100 tensiometer at room temperature (23±4° C.) and using surfactant free borate buffered saline as the probe solution. All equipment must be clean and dry; vibrations must be minimal around the instrument during testing. Wettability is usually reported as the advancing contact angle (Kruss DCA). The tensiometer was equipped with a humidity generator, and a temperature and humidity gage was placed in the tensiometer chamber. The relative humidity was maintained at 70±5%. The experiment was performed by dipping the lens specimen of known perimeter into the packing solution of known surface tension while measuring the force exerted on the sample due to wetting by a sensitive balance. The advancing contact angle of the packing solution on the lens is determined from the force data collected during sample dipping. The receding contact angle is determined from force data while withdrawing the sample from the liquid. The Wilhelmy plate method is based on the following formula: Fg=γρ cos θ−B, wherein F=the wetting force between the liquid and the lens (mg), g=gravitational acceleration (980.665 cm/sec²), γ=surface tension of probe liquid (dyne/cm), ρ=the perimeter of the contact lens at the liquid/lens meniscus (cm), θ=the dynamic contact angle (degree), and B=buoyancy (mg). B is zero at the zero depth of immersion. Typically, a test strip was cut from the central area of the contact lens. Each strip was approximately 5 mm in width and 14 mm in length, attached to a metallic clip using plastic tweezers, pierced with a metallic wire hook, and equilibrated in packing solution for at least 3 hours. Then, each sample was cycled four times, and the results were averaged to obtain the advancing and receding contact angles of the lens. Typical measuring speed was 12 mm/min. Samples were kept completely immersed in packing solution during the data acquisition and analysis without touching the metal clip. Values from five individual lenses were averaged to obtain the reported advancing and receding contact angles of the experimental lens.

Wettability of lenses was determined using a sessile drop technique using KRUSS DSA-100 TM instrument at room temperature and using deionized water as probe solution (Sessile Drop). The lenses to be tested were rinsed in deionized water to remove carry over from packing solution. Each test lens was placed on blotting lint free wipes which were dampened with packing solution. Both sides of the lens were contacted with the wipe to remove surface water without drying the lens. To ensure proper flattening, lenses were placed “bowl side down” on the convex surface of contact lens plastic molds. The plastic mold and the lens were placed in the sessile drop instrument holder, ensuring proper central syringe alignment. A 3 to 4 microliter drop of deionized water was formed on the syringe tip using DSA 100-Drop Shape Analysis software ensuring the liquid drop was hanging away from the lens. The drop was released smoothly on the lens surface by moving the needle down. The needle was withdrawn away immediately after dispensing the drop. The liquid drop was allowed to equilibrate on the lens for 5 to 10 seconds, and the contact angle was measured between the drop image and the lens surface. Typically, three to five lenses were evaluated, and the average contact angle reported.

The mechanical properties of the contact lenses were measured by using a tensile testing machine such as an Instron model 1122 or 5542 equipped with a load cell and pneumatic grip controls. Minus one diopter lens is the preferred lens geometry because of its central uniform thickness profile. A dog-bone shaped sample cut from a −1.00 diopter power lens having a 0.522 inch length, 0.276 inch “ear” width and 0.213 inch “neck” width was loaded into the grips and elongated at a constant rate of strain of 2 inches per minute until it breaks. The center thickness of the dog-bone sample was measured using an electronic thickness gauge prior to testing. The initial gauge length of the sample (Lₒ) and sample length at break (L_{f}) were measured. At least five specimens of each composition were measured, and the average values were used to calculate the percent elongation to break: percent elongation=[(L_{f}−Lₒ)/Lₒ]×100. The tensile modulus (M) was calculated as the slope of the initial linear portion of the stress-strain curve; the units of modulus are pounds per square inch or psi. The tensile strength (TS) was calculated from the peak load and the original cross-sectional area: tensile strength=peak load divided by the original cross-sectional area; the units of tensile strength are psi. Toughness was calculated from the energy to break and the original volume of the sample: toughness=energy to break divided by the original sample volume; the units of toughness are in-lbs/in³. The elongation to break (ETB) was also recorded as the percent strain at break.

The invention is now described with reference to the following examples. Before describing several exemplary embodiments of the invention, it is to be understood that the invention is not limited to the details of construction or process steps set forth in the following description. The invention is capable of other embodiments and of being practiced or being carried out in various ways.

The invention is now described with reference to the following examples. Before describing several exemplary embodiments of the invention, it is to be understood that the invention is not limited to the details of construction or process steps set forth in the following description. The invention is capable of other embodiments and of being practiced or being carried out in various ways.

The following abbreviations will be used throughout the Examples and have the following meanings:
BC: base or back curve plastic mold made of PP, TT, Z, or blends thereof
FC: front curve plastic mold made of PP, TT, Z, or blends thereof
PP: polypropylene which is the homopolymer of propylene
TT: Tuftec which is a hydrogenated styrene butadiene block copolymer (Asahi Kasei Chemicals)
Z: Zeonor which is a polycycloolefin thermoplastic polymer (Nippon Zeon Co Ltd)
RMM: reactive monomer mixture
LED: light emitting diode
DMA: N, N-dimethylacrylamide (Jarchem)
HEMA: 2-hydroxyethyl methacrylate (Bimax)
DPPM: (diphenylphosphoryl)-(4-vinylphenyl)-methanone

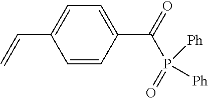 [SMILES: C=Cc1ccc(C(=O)P(=O)(c2ccccc2)c2ccccc2)cc1]

PVP K90: poly(N-vinylpyrrolidone) [CAS 9003-39-8] (Ashland)
mPDMS: mono-n-butyl terminated monomethacryloxypropyl terminated polydimethylsiloxane (Mₙ=800-1000 Daltons) (Gelest)
SiMAA: 2-propenoic acid, 2-methyl-2-hydroxy-3-[3-[1,3,3,3-tetramethyl-1-[(trimethylsilyl)oxy]disiloxanyl]propoxy]propyl ester (Toray) or 3-(3-(1,1,1,3,5,5,5-heptamethyltrisiloxan-3-yl)propoxy)-2-hydroxypropyl methacrylate
HO-mPDMS (n=4): mono-n-butyl terminated mono-(2-hydroxy-3-methacryloxypropyloxy)-propyl terminated polydimethylsiloxane (Ortec or DSM-Polymer Technology Group)

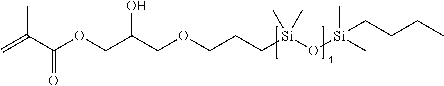

HO-mPDMS (n=15): mono-n-butyl terminated mono-(2-hydroxy-3-methacryloxypropyloxy)-propyl terminated polydimethylsiloxane (Mₙ=1400 Daltons) (Ortec or DSM-Polymer Technology Group)

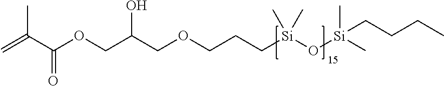

TEGDMA: tetraethylene glycol dimethacrylate (Esstech)
Omnirad 403: bis(2,6-dimethoxybenzoyl)-2,4,4-trimethylpentylphosphine oxide (IGM Resins)
Omnirad 1173: 2-hydroxy-2-methyl-1-phenylpropanone (IGM Resins)
Omnirad 1700: mixture of 25 weight % Omnirad 403 and 75 weight % Omnirad 1173 (IGM Resins)
Omnirad 1870: mixture of 70 weight % Omnirad 403 and 30 weight % Omnirad 1173 (IGM Resins)
Omnirad 819: bis(2,4,6-trimethylbenzoyl)-phenylphosphine oxide [CAS 162881-26-7] (IGM Resins)
AIBN: azobisisobutyronitrile [CAS 78-67-1]
Norbloc: 2-(2′-hydroxy-5-methacrylyloxyethylphenyl)-2H-benzotriazole (Janssen)
RB247: 1,4-Bis[2-methacryloxyethylamino]-9,10-anthraquinone [CAS #109561-07-1]
IMT Blue: sodium 1-amino-4-((4-(2-bromoacrylamido)-2-sulfonatophenyl)amino)-9,10-dioxo-9,10-dihydroanthracene-2-sulfonate

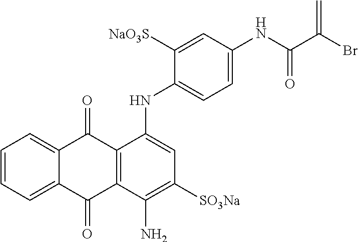

DIW: deionized water
IPA: isopropyl alcohol
3E3P: 3-ethyl 3-pentanol
D30: 3,7-dimethyl-3-octanol (Vigon)
PS: Borate Buffered Packing Solution: 18.52 grams (300 mmol) of boric acid, 3.7 grams (9.7 mmol) of sodium borate decahydrate, and 28 grams (197 mmol) of sodium sulfate were dissolved in enough deionized water to fill a 2-liter volumetric flask.
WC: water content (weight %)
EC Dk: edge-corrected oxygen gas permeability (barrers)
M: modulus (psi)
TS: tensile strength (psi)
ETB: elongation to break (%)
RI: refractive index
Sessile Drop: advancing contact angle (degrees)
mm: millimeter(s)
cm: centimeter(s)
μm: micrometer(s)
nm: nanometer(s)
L: liter(s)
mL: milliliter(s)
μL: microliter(s)
mW: milliwatt(s)
g: gram
mol: mole
g/mol: grams/mole
mg: milligram(s)
μg: microgram(s)
min: minute(s)
Da or Dalton(s): gram(s)/mole
kDa: kilodalton(s)

### Example 1—MAPO Deactivation and Subsequent Grafting

A RMM was prepared composed of 77 weight percent of the formulation listed in Table 1 and 23 weight percent of the diluent D30. The RMM was then filtered through a 3 μm filter using a stainless-steel syringe under pressure and degassed by applying vacuum (about 40 mm Hg). Under a nitrogen gas atmosphere and about 0.5 percent oxygen gas, about 75 μL of the reactive monomer mixture were dosed into the FC made of Zeonor. The BC made of Zeonor was then placed onto the FC, thereby forming a minus one diopter lens mold assembly. Pallets, each containing eight lens mold assemblies, were conveyed through a curing tunnel during which the pallets were irradiated for a total of about 10 minutes at 65° C. using 435 nm LED lights having intensity of about 4.5 mW/cm² at the pallet's surface. The light source was located above the pallets. BC were mechanically removed under yellow lighting. Pallets holding the FC with adhered lenses were stored under a nitrogen gas atmosphere and in the dark until further use.

TABLE 1
Formulation Components

| Component | Weight Percent |
|---|---|
| mPDMS | 31 |
| SiMAA | 28 |
| DMA | 24 |
| HEMA | 6 |
| PVP K90 | 7 |
| TEGDMA | 1.65 |
| Norbloc | 2 |
| RB247 | 0.01 |
| Omnirad 819 | 0.34 |

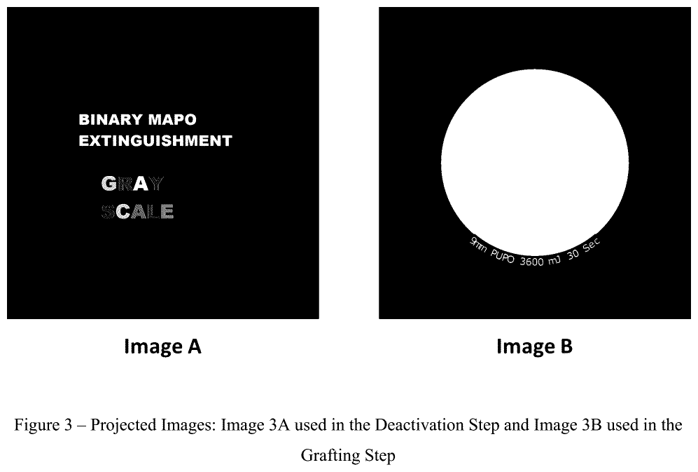

Working under yellow lights in a glove box, a FC with the lens still attached thereon was placed on the in-mold jig shown in FIG. 2 and attached to an optical lens forming apparatus equipped with a digital light projection unit employing a micro-mirror device to modulate and direct light onto the FC and attached lens. The light source of the apparatus was a 405 nanometer LED having an intensity of 53 mW/cm². The lens 1A on the in-mold jig was equilibrated under ambient conditions in air and then irradiated with Image 3A as shown in FIG. 3 for about 30 seconds. Another lens 1B on another in-mold jig was equilibrated under ambient conditions in air and then irradiated with Image 3A as shown in FIG. 3 for about 60 seconds. The imaged lenses and in-mold jigs were then placed in a glove box containing a nitrogen gas atmosphere to equilibrate under those inert conditions. Once equilibrated, about 150 microliters of a degassed 2.0 weight percent solution of IMT Blue in 50:50 (v/v) 1-propanol:DIW were dispensed on top of the lenses adhered to the FC on the in-mold jigs. After six minutes (enough time to allow the dye solution to diffuse into the lens but not long enough to impact the lens adhesion to the FC), the in-mold jigs were then irradiated under nitrogen with Image 3B (9-millimeter diameter spot with peripheral lettering) as shown in FIG. 3 for about 150 seconds, using the same 405 nanometer light source, thereby grafting the dye to the lenses in places having retained monoacylphosphine oxide groups.

The in-mold jigs were removed from the apparatus, and the lenses isolated by removing the FC mechanically. The lenses were then soaked in 70% (v/v) aqueous IPA for fifteen hours and then rinsed two times with DIW and two times with PS and then stored in vials in PS.

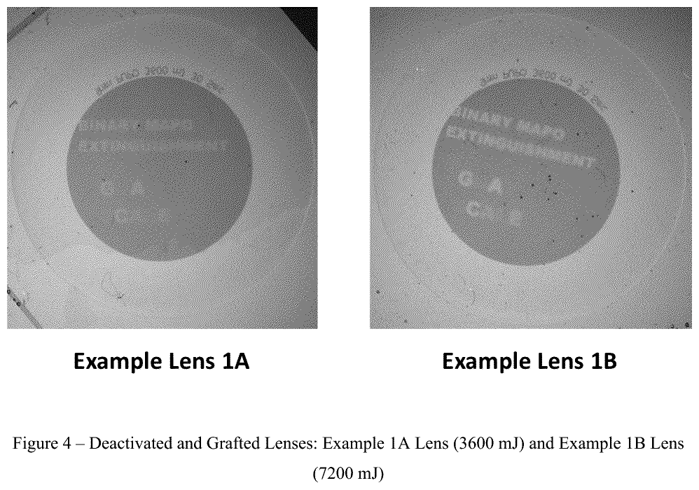

Micrographs of Example Lenses 1A and 1B are shown in FIG. 4 which demonstrate that deactivation of selective regions (Image 3A) was successful in both lenses, but the resolution Image 3A depends on the amount of deactivation energy applied and increased with increasing energy. Furthermore, the micrographs show that partially deactivated regions or greyscale regions can be created by modulating amount of deactivation energy within selective regions or across different selective regions. In any case, a wide range of patterns, markings, and barcode can be fabricated using the processes of deactivation followed by dye grafting.

### Example 2—Apodised Lens with Markings (Prophetic)

Example 1 is repeated except the RMM is composed of the formulation components listed in Table 2 without a diluent, that Image 3A comprising the numbers 123 is projected into the peripheral ring surrounding the optical zone of the lens, and that Image 3B is a spot image having an intensity gradient which increases linearly from the lens center to the lens edge.

TABLE 2
Formulation Components

| Component | Weight % |
|---|---|
| OH-mPDMS, (n = 4) | 54.89 |
| DMA | 19.54 |
| HEMA | 8 |
| PVP K90 | 12 |
| TEGDMA | 3.11 |
| Norbloc | 2.2 |
| Omnirad 819 | 0.25 |
| RB247 | 0.01 |

### Example 3—Thermally Cured Crosslinked Substrate Network Lens (Prophetic)

Example 2 is repeated except that the RMMs are shown in Table 3 and that crosslinked substrate network lenses are made by a thermal free radical copolymerization at a temperature between 60° C. and 100° C.

TABLE 3
Reactive Monomer Mixture

| | Example 3A | Example 3B |
|---|---|---|
| Component | Weight % | Weight % |
| OH-mPDMS, (n = 4) | 54 | 13 |
| OH-mPDMS, (n = 15) | 0 | 48 |
| DMA | 20 | 21 |
| HEMA | 8 | 6 |
| DPPM | 1 | 1 |
| PVP K90 | 11.7 | 7 |
| TEGDMA | 3 | 1.7 |
| Norbloc | 2 | 2 |
| AIBN | 0.3 | 0.3 |

1. An ophthalmic device formed by a process comprising:
a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
b) subjecting the reactive composition to a first activation step such that the reactive composition polymerizes therein to form a crosslinked substrate network containing a covalently bound activatable free radical initiator;
c) deactivating at least a portion of the covalently bound activatable free radical initiator at one or more selective regions of the crosslinked substrate network such that the crosslinked substrate network contains retained covalently bound activatable free radical initiator outside of the one or more selective regions;
d) contacting the crosslinked substrate network with a grafting composition containing one or more ethylenically unsaturated compounds, wherein the contacting is conducted under conditions such that the grafting composition penetrates into the crosslinked substrate network; and
e) activating the retained covalently bound activatable free radical initiator such that the grafting composition polymerizes with the crosslinked substrate network, thereby forming grafted polymeric networks outside of the selective regions.

2. The ophthalmic device of claim 1 wherein the deactivating of step (c) varies spatially within the ophthalmic device.

3. The ophthalmic device of claim 1 wherein the deactivating step (c) is achieved by irradiation at a wavelength capable of activating the initiator, wherein the initiation is conducted in an atmosphere containing oxygen gas.

4. The ophthalmic device of claim 1 wherein the grafting composition of step (d) contains a crosslinker.

5. The ophthalmic device of claim 1 wherein the grafting composition of step (d) is free of a crosslinker.

6. The ophthalmic device of claim 1 wherein in step (c), only a portion of the covalently bound activatable free radical initiator is inactivated at the selective regions such that step (e) includes forming a grafted network partially within the selective regions.

7. The ophthalmic device of claim 1 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

8. The ophthalmic device of claim 1 wherein the one or more ethylenically unsaturated compounds of step (d) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

9. The ophthalmic device of claim 1 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

10. The ophthalmic device of claim 1 wherein the polymerization initiator is a bisacylphosphine oxide or a bis(acyl)phosphane oxide.

11. The ophthalmic device of claim 1 that is in the form of a hydrogel and wherein the reactive composition contains one or more silicone-containing components, and the grafting composition contains one or more hydrophilic reactive components.

12. The ophthalmic device of claim 1 wherein the reactive composition, the grafting composition, or both the reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

13. The ophthalmic device of claim 1 further comprising: following step (e), contacting the crosslinked substrate network with a second grafting composition containing one or more ethylenically unsaturated compounds and activating retained covalently bound activatable free radical initiator such that the second grafting composition polymerizes with the crosslinked substrate network outside of the selective regions and optionally partially within the selective regions.

14. The ophthalmic device of claim 1 wherein steps (a) and (b) are performed in a mold assembly comprised of a front mold and a back mold, the front mold and the back mold defining and enclosing a cavity in the shape of the ophthalmic device therebetween, and process steps (c), (d), and (e) are performed in the mold assembly after the back mold has been removed.

15. The ophthalmic device of claim 1 wherein the ophthalmic device is selected from the group consisting of a contact lens, an intraocular lens, a punctal plug, and an ocular insert.

16. The ophthalmic device of claim 15 wherein the ophthalmic device is a contact lens or an intraocular lens.

17. An ophthalmic device that is a reaction product of a composition comprising:
a) a crosslinked substrate network containing covalently bound activatable free radical initiators outside of one or more selective regions and optionally partially within the one or more selective regions; and
b) a grafting composition containing one or more ethylenically unsaturated compounds, wherein the grafting composition is localized in the crosslinked substrate network where there are retained covalently bound activatable free radical initiators.

18. The ophthalmic device of claim 17 wherein the concentration of the retained covalently bound activatable free radical initiators varies spatially within crosslinked substrate network.

19. The ophthalmic device of claim 17 wherein the crosslinked substrate network is the reaction product of a reactive composition comprising: (i) a polymerization initiator that is capable, upon a first activation, of forming two or more free radical groups, at least one of which is further activatable by subsequent activation; (ii) one or more ethylenically unsaturated compounds; and (iii) a crosslinker.

20. The ophthalmic device of claim 19 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

21. The ophthalmic device of claim 20 wherein the polymerization initiator is a bisacylphosphine oxide or a bis(acyl)phosphane oxide.

22. The ophthalmic device of claim 17 wherein the covalently bound activatable free radical initiators are selected from the group consisting of monoacylphosphine oxides, bisacylphosphine oxides, and combinations thereof.

23. The ophthalmic device of claim 17 wherein the crosslinked substrate network is formed by the thermal free radical polymerization of a reactive monomer mixture comprising at least one reactive component having at least one pendant group selected from the group consisting of monoacylphosphine oxide, bisacylphosphine oxide, and combinations thereof.

24. The ophthalmic device of claim 17 wherein the one or more ethylenically unsaturated compounds in the grafting composition and the reactive composition comprise polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

25. The ophthalmic device of claim 17 wherein the crosslinked substrate network is formed from one or more silicone-containing components and the grafting composition contains hydrophilic reactive components.

26. The ophthalmic device of claim 17 wherein the crosslinked substrate network is formed from one or more hydrophilic reactive components and the grafting composition contains one or more silicone-containing components.

27. The ophthalmic device of claim 17 wherein the crosslinked substrate network is formed from one or more hydrophilic reactive components and the grafting composition contains hydrophilic reactive components.

28. The ophthalmic device of claim 17 wherein the crosslinked substrate network is formed from one or more silicone-containing components and the grafting composition contains one or more silicone-containing components.

29. The ophthalmic device of claim 17 wherein the crosslinked substrate network, the grafting composition, or both the crosslinked substrate network and the grafting composition contain one or more additives selected from UV absorbers, photochromic compounds, pharmaceutical compounds, nutraceutical compounds, antimicrobial compounds, tints, pigments, dyes, dyes, release agents, and wetting agents.

30. The ophthalmic device of claim 17 wherein the ophthalmic device is selected from the group consisting of a contact lens, an intraocular lens, a punctal plug, and an ocular insert.

31. The ophthalmic device of claim 30 wherein the ophthalmic device is a contact lens or an intraocular lens.

32. A process for making an ophthalmic device, the process comprising:
a) providing a reactive composition containing: (i) a polymerization initiator that is capable, upon a first activation, of forming two or more free radical groups, at least one of which is further activatable by subsequent activation; (ii) one or more ethylenically unsaturated compounds; and (iii) a crosslinker;
b) subjecting the reactive composition to a first activation step such that the reactive composition polymerizes therein to form a crosslinked substrate network containing a covalently bound activatable free radical initiator;
c) deactivating at least a portion of the covalently bound activatable free radical initiator at one or more selective regions of the crosslinked substrate network such that the crosslinked substrate network contains retained covalently bound activatable free radical initiator outside of the one or more selective regions;
d) contacting the crosslinked substrate network with a grafting composition containing one or more ethylenically unsaturated compounds, wherein the contacting is conducted under conditions such that the grafting composition penetrates into the crosslinked substrate network; and
e) activating the retained covalently bound activatable free radical initiator such that the grafting composition polymerizes with the crosslinked substrate network, thereby forming grafted polymeric networks outside of the selective regions.

33. The ophthalmic device of claim 32 wherein the deactivating of step (c) varies spatially within the ophthalmic device.

34. The ophthalmic device of claim 32 wherein the deactivating step (c) is achieved by irradiation in an oxygen gas atmosphere.

35. The process of claim 32 wherein the grafting composition of step (d) contains a crosslinker.

36. The process of claim 32 wherein the grafting composition of step (d) is free of a crosslinker.

37. The process of claim 32 wherein in step (c), only a portion of the covalently bound activatable free radical initiator is inactivated at the selective regions such that step (e) includes forming a grafted network partially within the selective regions.

38. The process of claim 32 wherein the one or more ethylenically unsaturated compounds of step (a) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

39. The process of claim 32 wherein the one or more ethylenically unsaturated compounds of step (e) comprise one or more polymerizable groups independently selected from: (meth)acrylate, (meth)acrylamide, styryl, vinyl, N-vinyl lactam, N-vinylamide, O-vinylether, O-vinylcarbonate, O-vinylcarbamate, C₂₋₁₂ alkenyl, C₂₋₁₂ alkenylphenyl, C₂₋₁₂ alkenylnaphthyl, and C₂₋₆ alkenylphenyl-C₁₋₆ alkyl.

40. The process of claim 32 wherein the polymerization initiator is a bisacylphosphine oxide, a bisacylphosphane oxide, a di-azo compound, a di-peroxide compound, an azo-bis(monoacylphosphine oxide), an azo-bis(monoacylphosphane oxide), a peroxy-bis(monoacylphosphine oxide), a peroxy-bis(monoacylphosphane oxide), an azo-bis(alpha-hydroxy ketone), a peroxy-bis(alpha-hydroxy ketone), an azo-bis(1,2-diketone), a peroxy-bis(1,2-diketone), a germanium based compound, tert-butyl 7-methyl-7-(tert-butylazo)peroxyoctanoate, or combinations thereof.

41. The process of claim 40 wherein the polymerization initiator is a bisacylphosphine oxide or a bis(acyl)phosphane oxide.

42. The process of claim 32 that is in the form of a hydrogel and wherein the reactive composition contains one or more silicone-containing components, and the grafting composition contains one or more hydrophilic reactive components.

43. The process of claim 32 wherein the reactive composition, the grafting composition, or both the reactive composition and the grafting composition contain one or more additives selected from UV absorbers, photochromic compounds, pharmaceutical compounds, nutraceutical compounds, antimicrobial compounds, reactive tints, pigments, copolymerizable dyes, non-polymerizable dyes, release agents, wetting agents, and release agents.

44. The process of claim 32 wherein the process further comprises: following step (e), contacting the crosslinked substrate network with a second grafting composition containing one or more ethylenically unsaturated compounds and activating retained covalently bound activatable free radical initiator such that the second grafting composition polymerizes with the crosslinked substrate network outside of the selective regions and optionally partially within the selective regions.

45. The process of claim 32 wherein the process steps (a) and (b) are performed in a mold assembly comprised of a front mold and a back mold, the front mold and a back mold defining and enclosing a cavity in the shape of the ophthalmic device therebetween, and process steps (c), (d), and (e) are performed in the mold assembly after the back mold has been removed.

46. The ophthalmic device of claim 1 wherein the selective regions form a pattern.

47. The ophthalmic device of claim 1 wherein the selective regions form a fiduciary marker.

48. The ophthalmic device of claim 1 wherein the selective regions form a barcode.

49. The ophthalmic device of claim 17 wherein the selective regions form a pattern.

50. The ophthalmic device of claim 17 wherein the selective regions form a fiduciary marker.

51. The ophthalmic device of claim 17 wherein the selective regions form a barcode.
